(19) United States
(12) Patent Application Publication
(10) Pub. No.: US 2026/0338689 A1
(43) Pub. Date: Oct. 29, 2026

(54) SELF-TUNING BUBBLE DETECTOR ASSEMBLIES, CALIBRATION METHODS, AND COMPUTER PROGRAM PRODUCTS FOR DIAGNOSTIC ANALYZERS

(71) Applicant: Siemens Healthcare Diagnostics Inc., Tarrytown, NY (US)

(72) Inventor: Ari Rosenfeld, Passaic, NJ (US)

(73) Assignee: Siemens Healthcare Diagnostics Inc., Tarrytown, NY (US)

(21) Appl. No.: 19/111,741

(22) PCT Filed: Sep. 14, 2023
(86) PCT No.: PCT/US2023/074199

§ 371 Date: Mar. 13, 2025

Related U.S. Application Data
(60) Provisional application No. 63/375,851, filed on Sep. 15, 2022.

(51) Int. Cl. G01N 21/61

(52) U.S. Cl. CPC G01N 21/61; G01N 2201/12769

(57) ABSTRACT

A self-tuning optical bubble detector assembly for diagnostic analyzers. The bubble detector assembly has a light emitter operated at a plurality of inputs (i.e., generating a plurality of light intensities) for dry supply line tubing (i.e., having no liquid) and for a wet supply line tubing (i.e., having liquid therein). A plurality of outputs representing light detected through the dry supply line tubing and the wet supply line tubing are generated. Based on the received outputs, a final calibrated setting (e.g., a % duty setting or an I_LED) and a threshold are determined at which to accurately and repeatedly determine if the supply line tubing has liquid or air therein. Further provided are methods of self-tuning an optical bubble detector for diagnostic analyzers and computer program products, as are other aspects.

### CROSS REFERENCE TO RELATED APPLICATION

This application claims the benefit of U.S. Provisional Patent Application No. 63/375,851, entitled “SELF-TUNING BUBBLE DETECTOR ASSEMBLIES, CALIBRATION METHODS, AND COMPUTER PROGRAM PRODUCTS FOR DIAGNOSTIC ANALYZERS” filed Sep. 15, 2022, the disclosure of which is hereby incorporated by reference in its entirety for all purposes.

### FIELD

This disclosure relates to diagnostic analyzers, and more particularly to bubble detector assemblies and methods used in such diagnostic analyzers.

### BACKGROUND

A wide variety of medical diagnostic analyzers (e.g., chemical analyzers and immunoassay instruments) are used to analyze patient specimens, such as, e.g., whole blood, blood serum or plasma, cerebrospinal liquid, interstitial fluid, urine, sperm, sputum, saliva, and the like. These diagnostic analyzers typically use one or more liquid reagents, wash liquids, or other process liquids in conjunction with processing of such patient specimens. In order to obtain accurate diagnostic results, a very precise amount of liquid reagent and possibly one or more other liquids may need to be transferred from one location (e.g., storage container or liquid supply) to a receptacle where a chemical reaction can take place, or vice versa. In many cases, the liquid transfer takes place through a probe (pipette) that is coupled to a length of flexible supply line tubing. Although bubble detectors are used to detect the presence of a liquid in tubing, such liquid detections may have certain performance shortcomings.

### SUMMARY

According to some embodiments, the disclosure provides and optical bubble detector assembly, comprising a controller configured to: operate a light emitter projecting light into a supply line tubing and detecting the transmitted light though the tubing by a light detector at a plurality of light intensities corresponding to a plurality of inputs; receive a first plurality of outputs from the light detector of the optical bubble detector, the first plurality of outputs corresponding respectively to the plurality of inputs, each of the first plurality of outputs representing an amount of light detected through the supply line tubing having no liquid therein; receive a second plurality of outputs from the light detector, the second plurality of outputs corresponding respectively to the plurality of inputs, each of the second plurality of outputs representing an amount of light detected through the supply line tubing having liquid therein; and select a particular one of the plurality of inputs as a final calibrated setting based upon selected ones of the first and second pluralities of outputs.

According to second embodiment, the disclosure describes a method of calibrating an optical bubble detector. The method comprises receiving a first plurality of outputs each representing an amount of light detected through a supply line tubing having no liquid therein, the first plurality of outputs corresponding respectively to a plurality of inputs to a light emitter; receiving a second plurality of outputs each representing an amount of light detected through the supply line tubing having a liquid therein, the second plurality of outputs corresponding respectively to the plurality of inputs to the light emitter; and selecting a particular one of the plurality of inputs as a final calibrated setting based upon selected ones of the first and second pluralities of outputs.

According to a third aspect, a computer program product is provided. The computer program product includes a non-transitory medium readable by a computer, the computer readable medium having computer program code configured to: receive a first plurality of outputs each representing an amount of light transmitted through a supply line tubing having no liquid therein for a respective plurality of inputs to a light emitter; receive a second plurality of outputs each representing an amount of light transmitted through the supply line tubing having a liquid therein for the respective plurality of inputs; and set a final calibrated setting based on selected ones of the first plurality of outputs and the second plurality of outputs.

Still other aspects, features, and advantages of the disclosure may be readily apparent from the following detailed description wherein a number of example embodiments are described and illustrated, including the best mode contemplated for carrying out the disclosure. The disclosure may also be capable of other and different embodiments, and its several details may be modified in various respects, all without departing from the scope of the disclosure. Accordingly, the drawings and descriptions are to be regarded as illustrative in nature, and not as restrictive. This disclosure covers all modifications, equivalents, and alternatives falling within the scope of the claims.

### BRIEF DESCRIPTION OF DRAWINGS

The drawings, described below, are for illustrative purposes only, and are not necessarily drawn to scale. The drawings are not intended to limit the scope of the disclosure in any way.

FIG. 1 illustrates a perspective view of a bubble detector assembly according to a first embodiment.

FIG. 2 illustrates a detailed schematic diagram of a first self-tuning bubble detector assembly configured to read a bubble detector according to embodiments.

FIG. 3A illustrates a schematic overview diagram of a second self-tuning bubble detector assembly configured to read a bubble detector according to embodiments.

FIG. 3B illustrates a detailed schematic diagram of the second self-tuning bubble detector assembly configured to read a bubble detector according to embodiments.

FIG. 4A illustrates a first graph of output voltage from a light detector versus % duty setting provided to drive a light emitter of a first embodiment of bubble detector assembly according to embodiments.

FIG. 4B illustrates a second graph of current to a light emitter versus reference voltage output from a light detector for a second embodiment of bubble detector assembly according to embodiments.

FIG. 5 illustrates a summary flowchart of a method of calibrating an optical bubble detector according to embodiments.

FIG. 6A illustrates a flowchart of a generic self-calibration method of calibrating an optical bubble detector (e.g., the first and second optical bubble detector of first and second optical bubble detector assemblies) according to embodiments.

FIG. 6B illustrates a flowchart of a first self-calibration method of calibrating a first optical bubble detector according to embodiments.

FIG. 6C illustrates a flowchart of a second self-calibration method of calibrating a second optical bubble detector according to embodiments.

FIG. 6D illustrates a flowchart of a method of generating DRY and WET calibration data for DRY and WET calibration curves for the second bubble detector calibration method according to embodiments.

FIG. 6E illustrates a flowchart of a method of finding I_LED_Final (the final calibrated setting) of the second bubble detector method according to embodiments.

FIG. 6F illustrates a flowchart of a method of determining V_Ref_Final of the second bubble detector method according to embodiments.

FIG. 7 illustrates views of Manchester auto-calibration command waveforms that can be used by the bubble detector assemblies according to embodiments.

FIGS. 8A and 8B illustrate zoomed in, and zoomed out views, respectively, of the auto-calibration command waveforms that can be used by the second self-tuning bubble detector assembly according to embodiments.

FIGS. 9A through 9F illustrate various views of the second bubble detector assembly and components thereof according to embodiments.

### DETAILED DESCRIPTION

Reference will now be made in detail to the example embodiments of this disclosure, which are illustrated in the accompanying drawings. Wherever possible, the same reference numbers will be used throughout the drawings to refer to the same or like parts.

When managing the flow of liquids, an apparatus such as a bubble detector is often used to ensure that the intended volume of liquid has actually been moved from one location to another. This is often performed with non-contact techniques that may include optical technologies. Basic and conditioned optical liquid/air sensors (referred to as “bubble detectors” herein) are known, but they may have limited performance with respect to diagnostic analyzers for medical applications. In particular, a need exists to provide optical bubble detector assemblies and/or methods of calibration of optical bubble detectors that can accurately and repeatedly detect the presence of certain liquids or air in supply line tubing. Supply line tubing, as used herein, means any tubing that is used to transfer a liquid from one location to another. In some embodiments, precise amounts of a small volume of the liquid in the supply line tubing used in such medical diagnostic analyzers can be measured. In other applications, the existence or absence of the liquid at certain locations along the supply line tubing can be determined, i.e., whether WET (containing a liquid) or DRY (containing predominantly air).

In such medical diagnostic analyzers, probes (e.g., pipettes) coupled to, and moveable by, a robot are often used to aspirate a liquid, such as a patient specimen liquid and/or reagent liquid and transfer the specimen and/or reagent liquid to another location and then dispense the liquid(s) into a receptacle, such as a cuvette. After which, the liquids may undergo further processing and/or incubation. After the transfer and dispense of the liquid(s), the probe can be washed in some instances so as to reduce carryover of the liquid(s) to a next diagnostic test. In some embodiments, washing can be accomplished by lowering the probe and aspirating a wash liquid into the probe at a wash station having a receptacle or well containing the wash liquid.

In another application, such as at a wash station of a diagnostic analyzer, a wash station probe may be used to accomplish washing of magnetic beads contained in a vessel as part of the incubation process that is taking place on the diagnostic analyzer. The wash operation can take place at a wash station such as on a wash or incubation ring of the diagnostic analyzer, for example. In some embodiments, more than one such wash station can be provided on the diagnostic analyzer. In some embodiments, wash liquid may be dispensed from the probe through the supply line tubing. In other embodiments, process and/or wash liquid may be aspirated into the probe and supply line tubing. In each case, in order to ensure that the dispense, aspiration, and/or wash operation has indeed taken place, the inventors herein have included a bubble detector assembly located along and coupled to a length of the supply line tubing that is coupled to the probe. Such a bubble detector assembly may be located at a location where the liquid or air determination or measurement is sought, such as near or even directly adjacent to the probe or elsewhere along the supply line tubing. Thus, the bubble detector assembly according to embodiments described herein can be configured to ensure that an aspiration and/or dispense of a liquid (wash liquid, liquid reagent, or even a specimen) has successfully occurred.

Therefore, embodiments of bubble detector assemblies, self-calibrating electronic circuits thereof, self-calibration methods, and computer program products disclosed herein may improve the performance of such bubble detector assemblies making them much more practical for use in demanding fluid flow monitoring applications, and in particular for uses in medical diagnostic analyzer applications, especially where subtle transitions between very clear liquids versus passing air are being detected.

In particular, in reagent liquid dispense operations, it is desired that a precise amount of liquid reagent be transferred from a storage container to the receptacle (e.g., cuvette) where chemical reactions will take place facilitating quantitative measurement of a specific analyte or substance contained in a specimen. In wash operations, one or more wash liquids may be introduced into fluidic manifolds and/or supply line tubing in order to conduct wash operations of magnetic beads contained in a cuvette. Such wash operations may remove process and/or wash liquids through supply line tubing, and/or clean all wetted surfaces of a reagent probe or wash probe. This can minimize or avoid carryover effects from one test to another, and/or facilitate wash processes. In one aspect, the present method and assembly can detect that a wash operation has indeed been successfully conducted.

In some embodiments, a pump, such as a diluter pump may be used to transfer (e.g., aspirate and then dispense) these liquid reagents in quantities as small as 100 μL or even smaller. Due to the medical significance of these assays and chemical tests carried out by the diagnostic analyzers, in some embodiments, the delivery of a known volume of reagent should be carried out repeatedly with no significant errors. To this end, diagnostic analyzers may not rely merely on a pump's ability to dispense or aspirate known volumes of reagents via an open loop control. Rather, each dispense or aspiration of reagent should be checked for correctness of volume by one or more additional sensors, signal conditioning electronics, and/or combination of firmware and/or software methods.

Some known medical diagnostic analyzers, such as, e.g., a CENTAUR XP System available from SIEMENS HEALTHINEERS, use optical sensors for confirming that reagents are being properly delivered through the system's supply line tubing. For reagent probes, i.e., probes that are used for aspirating and dispensing liquid reagent, a bubble detector with a light emitter/photodetector pair has been used that enables the software to ascertain that the correct volume was aspirated by the reagent probe from a reagent container of the analyzer and then dispensed into the receptacle (e.g., cuvette) for incubation and analysis. Any suitable analyte or constituent detection method may be used by the diagnostic analyzer, such as using chemiluminescence, absorbance, or the like. For this type of reagent aspiration, a slug of reagent can be sandwiched with a leading air gap and trailing air gap surrounding the slug in the supply line tube.

Since the software is instructing the pump to aspirate and/or dispense the liquid reagent into and/or out of the reagent probe over a specific time interval, there is an expected timeframe over which the bubble detector will see air followed by liquid, and then followed by air. In this fashion, the software can detect if the correct aliquot of liquid reagent was indeed dispensed and, if not, generate a flag to indicate that something went wrong in the process. Potential causes of such a flag may be that the reagent ran out, that there is a blockage in the supply line tubing, and/or that a bubble was detected.

In the bulk fluid area of the diagnostic analyzer, presence of one of the reagents may also be detected using a bubble detector with a light emitter/light detector pair. For example, the bubble detector may be used to detect that a reagent container (e.g., bottle) has run out and that the supply line tubing has gone dry.

However, in these prior art systems, the calibration process that enables differentiation between a dry supply line tubing (DRY) and a wet supply line tubing (WET) containing the liquid (e.g., reagent or wash liquid, for example) is either manual or automatic through adjusting operating settings. And although somewhat adequate, the prior art systems and methods of calibration are less than robust and may take a fair amount of experimentation to get them properly calibrated. In particular, it is desirable that such systems be self-calibrating with minimal manual input for each slightly different assembly, as each may contain different structure and/or materials, or may change over time. Moreover, improved calibration is needed for systems where very clear liquids are aspirated and/or dispensed, such as in diagnostic analyzer applications, as the optical differences between such clear liquids and air are slight.

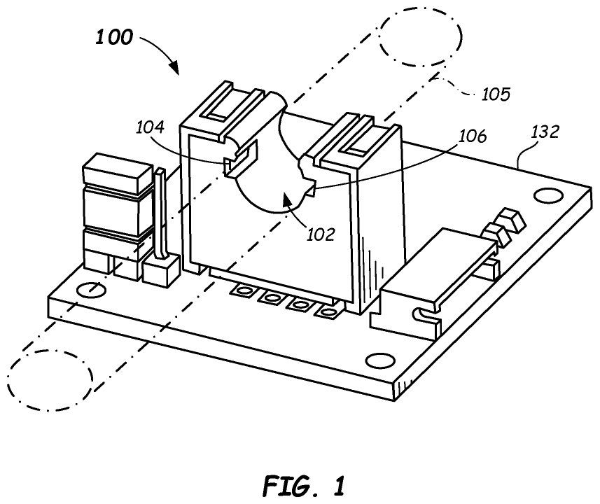

As shown in FIG. 1, a first embodiment of a bubble detector assembly 100 according to the present disclosure is shown. Bubble detector assembly 100 may have a channel 102 configured for receiving a supply line tubing 105 (shown dotted) therein. Bubble detector assembly 100 may have a light emitter, which may be an LED (light emitting diode) on one of sides 104 or 106, and may have a light detector, which may be a photodetector, on the other of the sides 104 or 106. When a new container (e.g., bottle or other suitable reservoir) of reagent liquid is installed on the diagnostic analyzer, a software counter may be set to a value that corresponds to the number of aspirations available for that container volume. With every aspiration, the counter may be decremented by one. Once the counter has reached zero, the operating software may not allow further tests to run and may prompt the operator, via a computer monitor, for example, to replace or refill the reagent container. The present disclosure includes a bubble detector assembly 100 including the light emitter/light detector pair at sides 104 and 106 that may act as a failsafe mechanism to the software counter method to ensure that a diagnostic test by the diagnostic analyzer does not run unless a suitable amount of the reagent liquid is indeed present. The bubble detector 100 may therefore provide a safety feedback mechanism above and beyond the software counter by detecting whether the reagent liquid is actually present in the supply line tubing 105. Other suitable uses for the bubble detector assembly 100 in diagnostic analyzers may include, but is not limited to, determining bulk reagent presence or absence, wash liquid presence or absence, process liquid presence or absence, and/or liquid measurement.

In the above application (e.g., the bulk reagent container application) light can be transmitted through the supply line tubing laterally (crosswise) from one side and detected on the other side of the supply line tubing 105 by the light detector (e.g., a photodiode). In some embodiments, the light emitter (e.g., LED) and light detector (e.g., photodetector) may be situated along opposing opposite sides of the same axis of the supply line tubing 105. In other embodiments described herein, the light emitter (e.g., LED) and light detector (e.g., photodetector) may be positioned on one side of the tubing axis, and may include a reflecting surface on the opposite side of the tubing axis.

According to the present disclosure, the signal conditioning circuitry that is configured to receive the analog signal from the light detector should be configured to be able to differentiate between a dry supply line tubing (DRY) and a wet supply line tubing (WET) containing the liquid (e.g., reagent, process, or wash liquid, for example). The detected output can be a function of such variables as, for example, the optical alignment of the light emitter (e.g., LED) and the light detector (e.g., photodetector), optical properties of the tubing due to pigmentation and size, turbidity or color of the liquid passing though the supply line tubing 105, and possibly other factors. Note that the bubble detector alignment may, and usually does, vary from bubble detector to bubble detector due to at least the mechanical misalignment of the light emitter with the light detector. The thickness and/or opacity of the tubing can also vary at various locations along a piece of the supply line tubing 105. These variations may shift the WET and DRY state output voltage by as much as half a volt. Thus, a robust calibration method is needed that can be used to properly calibrate each individual bubble detector assembly, as described herein.

Some known bubble detectors may have some degree of self-calibration, but it is generally too limited for use with at least some diagnostic analyzers. Such prior art self-calibration capability may simply establish a fixed operating output based upon experience with the bubble detector. On-board circuitry may implement intensity control of the light emitter using an adjustable current source. During calibration, a microcontroller may adjust the intensity of the light from the light emitter incrementally and read the emitted voltage of the light detector. In this fashion, the firmware seeks a current from one of the input settings that is capable of driving the light emitter to a desired set point. The calibrated setting can be saved in memory of the microcontroller so that any time it is powered up thereafter it will resume operation at this calibrated setting. This single case calibration may be useful in some applications, but is generally insufficient for diagnostic analyzer applications including very clear liquids.

Further, this prior art approach, however, does not consider the relationship between measured voltages indicating DRY and WET tubing, which is important for bubble detection in diagnostic analyzers that determine the existence of a particular liquid in the supply line tubing 105 or to otherwise quantify an amount of the liquid that has been aspirated and/or dispensed. In particular, a need exists for an improved calibration method in these applications, as the liquids (e.g., reagent and wash liquids) tend to be very clear, and thus the optical differences between WET and DRY readings can be very small and quite difficult to detect.

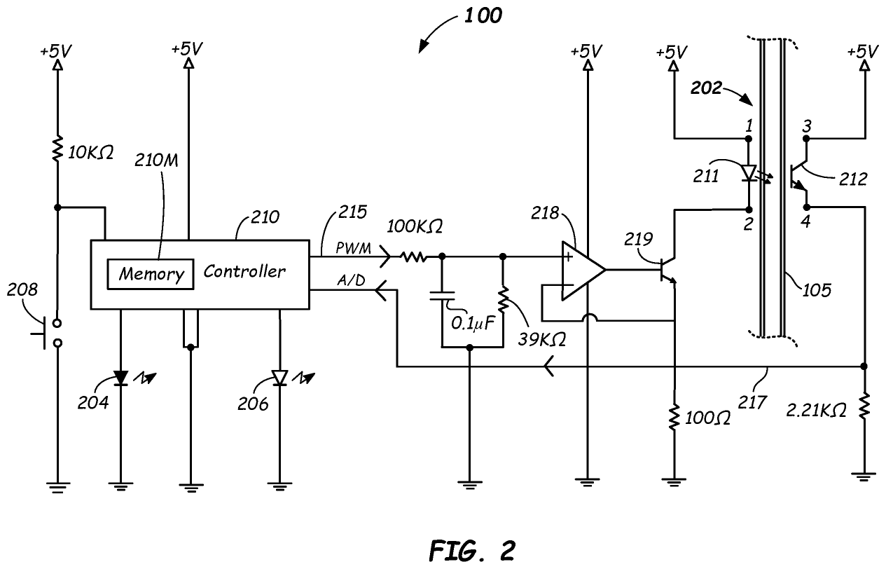

FIG. 2 shows a first embodiment of a bubble detector assembly 100 including a self-tuning electrical circuit according to embodiments of the disclosure. This bubble detector assembly 100 may be operated to optimize circuit performance (tuning) for a bubble detector 202 thereof, such as, e.g., the OPTEK OPB350W250Z bubble detector, in accordance with one or more embodiments herein. The bubble detector assembly 100, which can include one or more printed circuit board assemblies, can be used with probes (pipettes) where a liquid is aspirated, such as when the liquid is a liquid reagent or a wash liquid, but can be used in any analyzer (assay instrument or chemical analyzer) where determining the presence or absence of a liquid at a location along a supply line tubing 105 is desired.

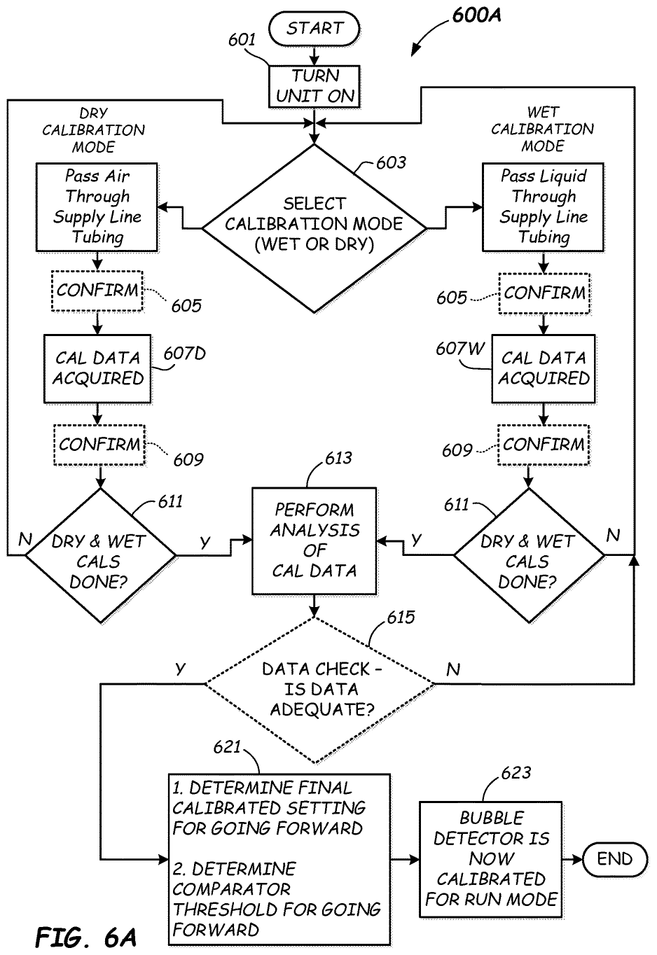

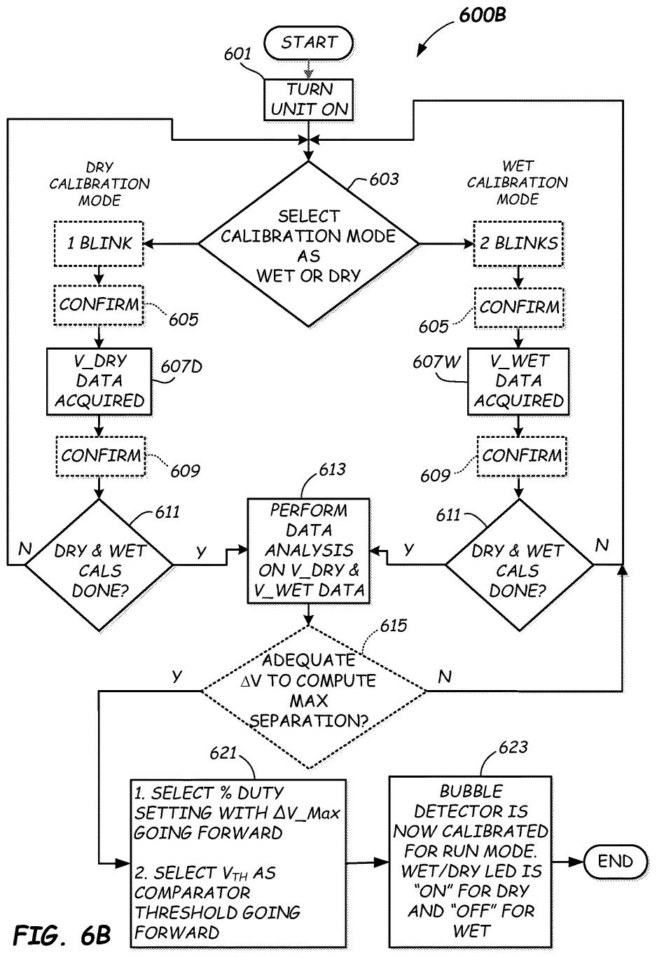

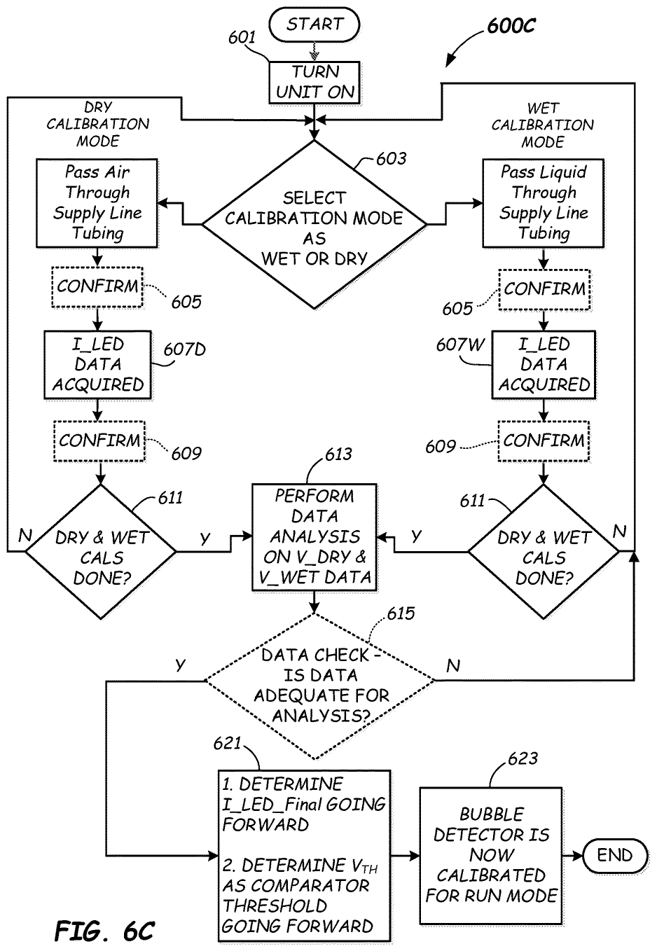

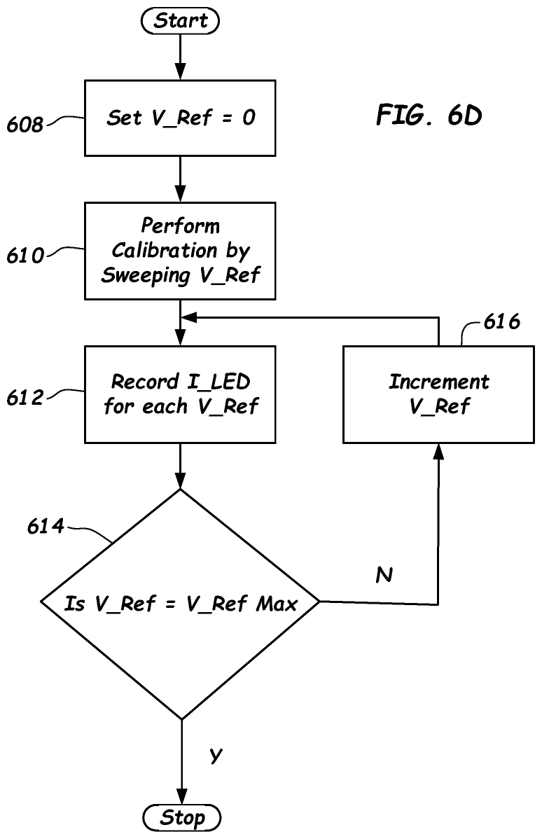

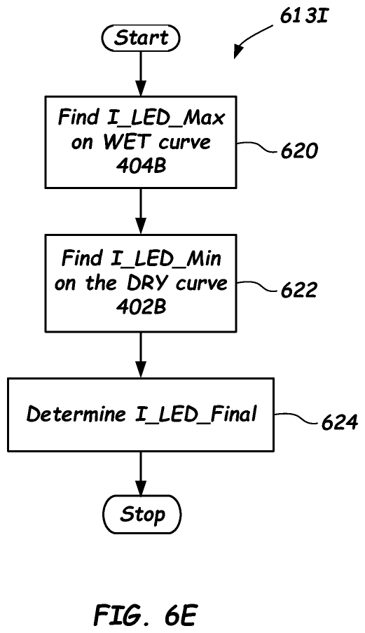

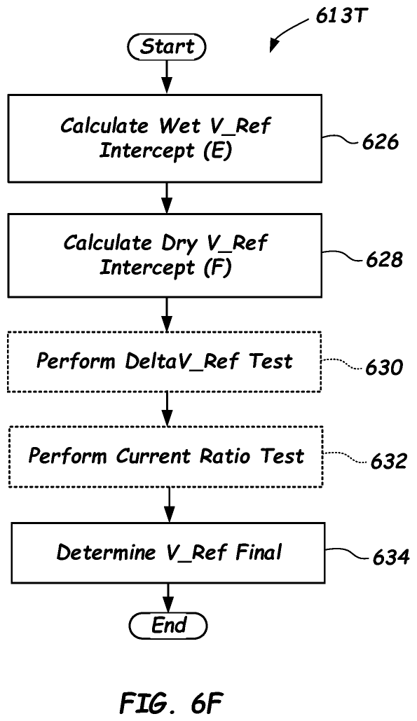

Given the variabilities stated above, tuning for each individual bubble detector 202 is desired. Self-tuning with the bubble detector assembly 100 described herein may include the use of a calibration LED 204 and a DRY/WET tubing indicator 206 (e.g., an LED), which are operable, respectively, to display a calibration state and a condition in the supply line tubing 105 (as either WET or DRY). To facilitate self-calibration, one or more push button inputs (e.g., push button 208) and one or more calibration outputs (e.g., LEDs) 204, 206 may be used. Following the flow chart presented in FIGS. 6A and 6B, the bubble detector assembly 200 can perform a calibration/self-tuning process in accordance with embodiments of the disclosure.

In the first embodiment, the calibration/self-tuning method may begin with the supply line tubing 105 in the DRY state. A section of the supply line tubing 105 (shown dotted) passes through the bubble detector 202. The supply line tubing 105 can be coupled to a pipette and optionally to a reagent or waste container. When a DRY “CALIBRATE” sequence is initiated, a controller 210 (which may be a PIC® microcontroller, such as, e.g., a PIC16F876 by Microchip Technology Inc.) may step through a full range of % duty settings supplied as inputs to the light emitter circuitry. The inputs may be pulse width modulated (PWM) signals (or waveforms) in input line 215, for example.

The LED intensity of a light emitter 211 can be controlled by three stages of electronics, for example. The first stage can be a PWM input from the controller 210. This input signal in line 215 can drive an RC circuit (the 2nd stage), converting the PWM output into a voltage. This voltage can drive an OP-AMP 218/transistor 219 pair (3rd stage) resulting in an adjustable current sink from the cathode terminal of the light emitter 211. The anode terminal of the light emitter 211 can be connected to the circuit's positive circuit supply. In this manner, each % duty setting of the PWM signal generated by the controller 210 corresponds to a different steady-state current and thus corresponds to a different intensity level emitted from the light emitter 211 (e.g., light emitting diode (LED)).

The PWM signal input in line 215 is injected into conditioning portions of the circuit of the bubble detector assembly 100 to generate a DC voltage at the input of the OP-AMP 218. OP-AMP 218 may be a TS922 rail-to-rail dual BiCMOS operational amplifier, for example. This signal can drive the transistor 219, which may be a bipolar junction transistor (BJT) creating a current sink for the light emitter 211. Transistor 219 may be a 2N222 NPN bipolar junction transistor, for example. Each % duty cycle of the PWM waveform is correlated with a specific sink current to the light emitter 211. The light signal that passes through the supply line tubing 105 is converted into a DC voltage at an output pin of a light detector 212. This detector output voltage in line 217 can be digitized with an A/D converter and stored in memory 210M of the controller 210. The DRY calibration involves injecting ever-increasing % duty settings of the PWM signals to generate a series of different light intensity levels. Each output (e.g., voltage) in output line 217 can correspond to a respective input of a % duty setting of the PWM signal.

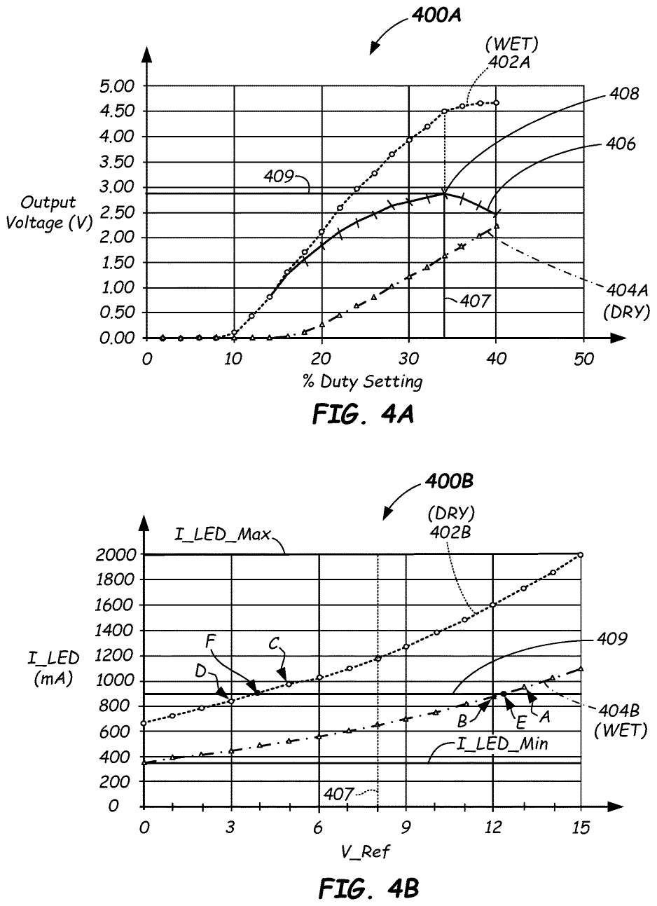

Next, the supply line tubing 105 may be made WET and the WET “CALIBRATE” sequence can be initiated, such as by pressing button 208. As was performed during the DRY “CALIBRATE” routine, the WET “CALIBRATE” injects the same set of % duty settings of PWM signals into the circuit to generate corresponding intensity levels of the light emitter 211 as will be explained below. The data forming the DRY and WET “CALIBRATE” curves (see 404A, 402A) are shown in FIG. 4A. These curves are different due to the altered light scattering that occurs between the DRY and WET calibration sequences due to the presence or absence of the liquid in the tube at the location of the bubble detector 202.

The supply line tubing 105 can be made WET by any suitable pump or vacuum supply coupled or interconnected to the supply line tubing 105 providing the liquid into the area of the supply line tubing 105 that receives the light signals from the light emitter 211. In the case of reagent aspiration and dispense, the supply line tubing 105 is made WET by aspirating a desired amount of the reagent liquid so that the reagent liquid is present between the light emitter 211 and the light detector 212. In the case of one wash operation, a wash liquid can be aspirated from a wash reservoir into which a pipette that is immersed therein. The wash liquid can be aspirated by operation of a pump until the wash liquid occupies a portion of the supply line tubing 105 that lies between the light emitter 211 and the light detector 212. In another wash embodiment, a wash probe can be lowered into a receptacle (e.g., cuvette) and used to aspirate process liquid, and/or wash magnetic beads with a wash liquid, wherein the light emitter 211 and light detector 212 can be positioned at a point along the supply line tubing 105 to determine if the liquid has been aspirated and/or dispensed into the supply line tubing 105. The light emitter 211 and the light detector 212 of the bubble detector 202 can be located directly adjacent to the probe or at another location along the supply line tubing 105.

In each embodiment, controller 210 may again walk through the same full range of the same % duty settings of the PWM signal as were previously input in input line 215 for the DRY case. The measured output (e.g., voltage) in output line 217 from the light detector 212 can be digitized at the output to the controller 210, as before. The digitized output voltage values resulting from each injected % duty setting can be stored in memory 210M and/or sent to an external computer for analysis.

According to embodiments, the bubble detector assembly 100 then performs an analysis to determine which particular one of the % duty settings (hereinafter the “final calibrated setting”) achieves excellent signal separation. In particular, it is desired to achieve a maximum separation difference (see line 409 of FIG. 4A) between the output voltages for the WET and DRY cases. In some embodiments, the final calibrated setting 407 can be a % duty setting input value that provides a maximum (largest) voltage difference between the recorded outputs (e.g., voltages) for the WET and DRY calibration sub-methods.

For example, in the so-called “maximum separation” embodiment (the first embodiment), the controller 210 may calculate a Vwet−Vdry voltage spread for each % duty setting provided as an input in line 215, and further search for a particular one of the % duty settings (hereinafter the “final calibrated setting”) that provides a maximum (largest 408) voltage spread. Curve 406 is the plot of the differences in output voltage in line 217 for each of the respective % duty settings. Curve 402A is the plot of the WET output voltages in line 217 for the WET calibration. Curve 404A is the plot of the DRY output voltages in line 217 for the DRY calibration. This particular final calibrated setting 407 may be stored in memory 210M, such as NVRAM (non-volatile random access memory). Going forward, the final calibrated setting 407 may be used to drive the light emitter 211 for excellent signal separation. Light emitter 211 may be any suitable light emitting device, such as a light emitting diode (LED). Light detector 212 may be any suitable photodetector.

In this embodiment, a midpoint voltage 409 between the Vdry and Vwet at that final calibrated setting 407 may be calculated and stored in memory 210M. This midpoint voltage 409 may be used, as a firmware comparator reference (i.e., a voltage threshold (V_{TH})) going forward to determine whether a supply line tubing 105 is WET, i.e., contains liquid, or is DRY, i.e., contains no liquid. DRY/WET tubing indicator 206 may illuminate when the V_{TH} at line 409 is met or exceeded thus signaling that a WET tubing condition is detected as shown in FIG. 4A. More practically, this output signal 217 to the controller 210 could be used to drive a digital I/O line of an external electronic circuit, microcontroller, or computer. The calculations may be carried out by the controller 210 and/or an external electronic circuit, microcontroller, or computer, or a combination thereof. These methods and assemblies may respond to the detected WET or DRY bubble detector state, such as by providing an error that can alert an operator. A flagged error can indicate that an improper state has been output for the particular operational stage of the process being undertaken, such as aspiration or dispense of a liquid reagent or aspiration or dispense of a wash liquid, for example.

In more detail in FIG. 2, bubble detector assembly 100 may include an operational amplifier 218 coupled to receive the input in input line 215 from controller 210, which may be a pulse-width modulated (PWM) signal. The operational amplifier 218, along with a transistor 219, may be configured to convert the PWM drive signal into an adjustable current and provide a current drive signal to light emitter 211 that is responsive to input in input line 215. This supplies variable current input to the light emitter 211 controlling the intensity of the light emitted by light emitter 211, which can be varied (e.g., in stepped increments) during the WET and DRY calibrations. After calibration, the input value corresponding to the final calibrated setting is used to illuminate the light emitter 211. Note that as used herein, each “% duty setting” is an input that is proportional to an intensity level (e.g., magnitude) of the light emitted by light emitter 211. For example, a 30% duty setting for the PWM signal may correspond to an intensity level of 30% of the maximum intensity of light emitter 211. The maximum separation calibration method described herein comprises selecting a % duty setting of the PWM signal to supply from the controller 210 and then a comparator reference V_{TH} for maximum WET-DRY output signal separation that can be tuned to establish the most “centered” setting.

FIG. 4A illustrates a graph 400A of example calibration data for calibration runs in accordance with the first embodiment, which seeks a maximum separation of WET and DRY signals. FIG. 4B illustrates graph 400B of example calibration data for calibration runs in accordance with a second embodiment, which seeks a maximum separation of WET and DRY signals by examining the maximum and minimum recorded values (the so-called ½ Max-Min embodiment) described herein below.

The test data in FIG. 4A includes output voltages of the light detector 212 versus % duty settings of the PWM inputs, which correspond to intensities of the light emitter 211 for a DRY tubing (Vwet data curve 402A) and a wet tubing (Vdry data curve 404A). The calibration data also illustrates voltage differences between respective voltages for the WET and DRY tubing conditions for each % duty setting (see ΔV=Vwet−Vdry data curve 406). In this example, 20 different % duty settings ranging from 0% to 40% duty were used to obtain the respective pluralities of Vdry and Vwet output voltages resulting in a Vwet data curve 402A and a Vdry data curve 404A. Other numbers and ranges of % duty settings may be used as inputs in other embodiments. For example, at least ten % duty settings ranging from 10% to 90% may be used. In this example, the % duty setting that produced the largest Vwet−Vdry signal separation is about 34%, as indicated by point 408 on the ΔV=Vwet−Vdry data curve 406.

As shown in FIG. 4A, at this % duty setting (the final calibrated setting) on line 407, the midpoint voltage between the Vwet and Vdry voltages is about 2.85 volts (see line 409). In some embodiments, this midpoint voltage may be set as the threshold voltage V_{TH} for determining whether the tubing condition is WET or DRY during actual non-calibration run-time, i.e., as a trigger point below which the supply line tubing 105 is determined to be DRY, and equal to or above which the supply line tubing 105 is determined to be WET. In other embodiments, the threshold voltage V_{TH} may be set to a voltage other than the midpoint between the Vwet and Vdry voltages at which the largest voltage difference ΔV occurs, such as slightly above or below the midpoint (e.g., +/−5%, for example).

In some embodiments, the bubble detector assembly 100 and firmware thereof may include a small circuit board 132 to which the bubble sensor 202 is mounted (e.g., in a manner similar to known sensors). In other embodiments, the bubble detector assembly 100 and firmware may be placed on one or more separate circuit boards if the bubble detector location on the supply line tubing 105 does not provide adequate space for the bubble detector assembly 100.

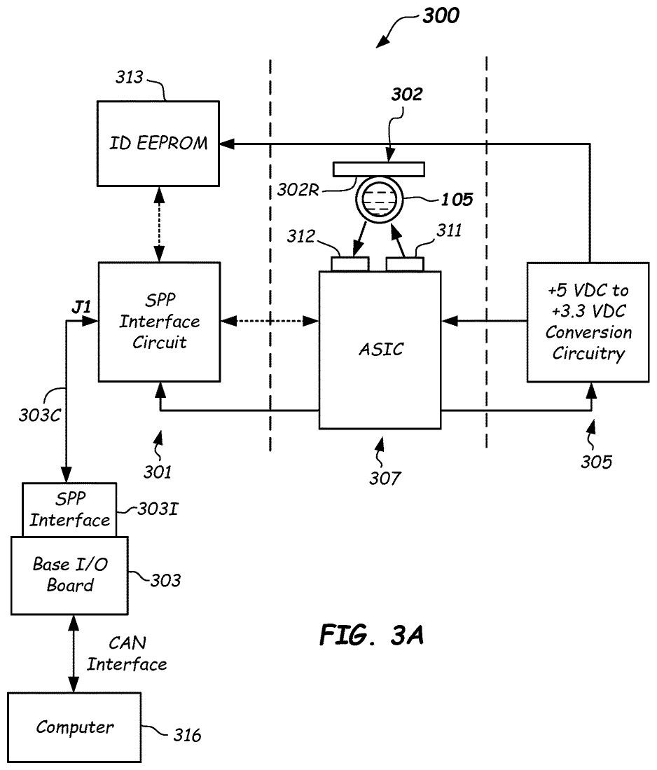

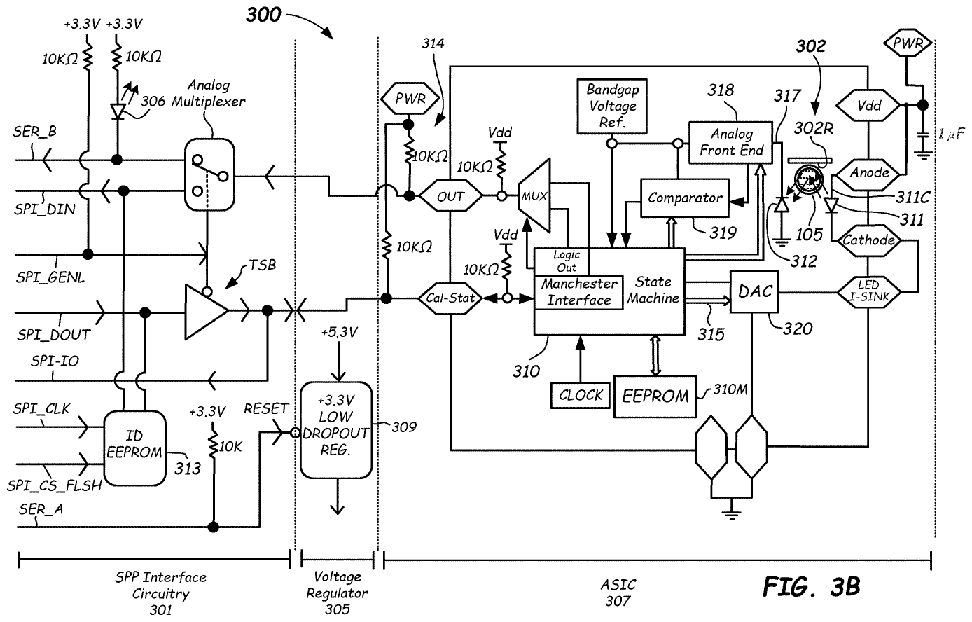

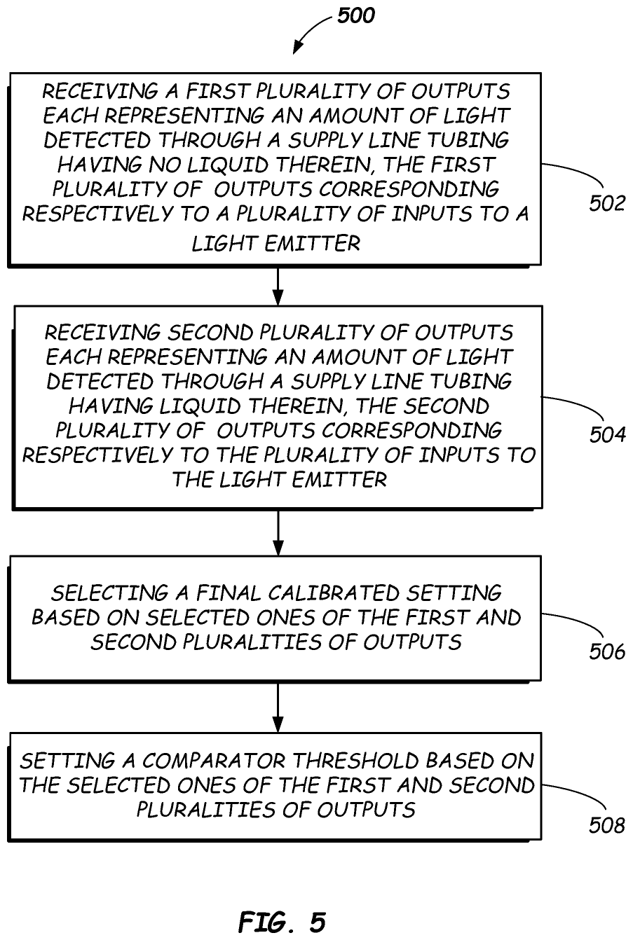

FIG. 5 illustrates a flowchart of a method 500 of calibrating an optical bubble detector in accordance with one or more embodiments disclosed herein, such as the embodiments of FIG. 2 and FIGS. 3A-3B. At process block 502, the method 500 may include receiving a first plurality of outputs (e.g., DRY voltages 404A in FIG. 2 (first embodiment) or DRY outputs in line 317 that may be proportional to DRY currents (I_LED) 402B in FIG. 4B (second embodiment)) each representing an amount of light detected through a supply line tubing (e.g., supply line tubing 105) having no liquid therein (i.e., the DRY calibration mode), the first plurality of outputs corresponding respectively to a plurality of inputs to a light emitter (e.g., light emitter 211, 311). For example, the inputs are % duty settings of the PWM signal in input line 215 for the embodiment of FIG. 2 (first embodiment), and inputs in line 315 to the DAC 320 (sweeping current until the comparator voltage of comparator 319 equals each V_Ref value) for the embodiment of FIG. 3B (second embodiment).

Again referring to FIG. 5, at process block 504, method 500 may include receiving a second plurality of outputs (e.g., WET voltages 402A in FIG. 2 (first embodiment) or WET output voltages in line 317 (proportional to currents (I_LED) 404B in FIG. 3B (second embodiment)) each representing an amount of light detected through the supply line tubing (e.g., supply line tubing 105) having a liquid (e.g., liquid reagent or wash liquid) therein (i.e., the WET calibration mode), the second plurality of outputs corresponding respectively to the plurality of inputs to the light emitter (e.g., light emitter 211, 311). For example, the inputs can be % duty settings in input line 215 for the embodiment of FIG. 2 (first embodiment), and inputs in line 315 that control current to the light emitter 311 via the DAC 320. Here the state machine 310 can sweep current to the light emitter 311 until the comparator voltages at comparator 319 are equal the V_Ref values) for the embodiment of FIG. 3B (second embodiment). To be clear, the same inputs are used for the DRY and WET calibrations.

At process block 506, the method 500 may include selecting a “final calibrated setting” based upon selected ones of the first and second pluralities of outputs. The selected ones can be selected from the respective pairs of DRY and WET outputs such that they achieve maximum signal separation. For example, in the first embodiment, a particular one of the plurality of % duty setting inputs in input line 215 of FIG. 2 is correlated with particular ones (pairs) of the plurality of outputs (output voltage pairs for the WET and DRY cases) in FIG. 2 (See also FIG. 4A). The selected ones can be respective outputs of pairs of DRY and WET voltage values at a particular % duty setting (the final calibrated setting). Likewise, in the second embodiment, a particular one of the plurality of voltage inputs in input line 315 of FIG. 3B is correlated with particular ones of the plurality of voltage outputs (corresponding to pairs of I_LED values for the DRY and WET cases—See FIG. 4B). The controller 210, 310 can be configured to operate the light emitter 211, 311 at the final calibrated setting at run-time for detecting whether the supply line tubing 105 contains the liquid (e.g., liquid reagent or wash liquid), i.e., (WET), or not (DRY). The selected final calibrated setting can be selected based on achieving a maximum difference ΔV between the WET and DRY outputs in the embodiment of FIG. 2 (first embodiment). The selected final calibrated setting can be based on a minimum value of the DRY curve and a maximum setting of the WET curve in FIG. 4B for the embodiment of FIGS. 3A-3B (second embodiment).

At process block 508, the method 500 may further include setting a threshold (e.g., V_{TH}) based on the selected ones of the first and second pluralities of outputs. The threshold can be selected to be located at the midpoint between the WET and DRY output levels for the final calibrated setting (e.g., between first voltages 404A and second voltages 402A of FIG. 2 and between first currents 402B and second currents 404B of FIG. 3B). In one example, for the first embodiment, the threshold voltage 409 may be set at a voltage value at which the largest voltage difference (ΔV Max) occurs between the first and second pluralities of voltages (i.e., the maximum difference at 408 in FIG. 4A).

In another embodiment, the controller (state machine) 310 and the method 500 can be operational, as shown in FIG. 3B and FIG. 4B, to select a maximum current (I_LED_Max) corresponding to the first plurality of outputs, and select a minimum current (I_LED_Min) corresponding to the second plurality of outputs, and then set the final calibrated setting 409 to a current value located between the maximum current (I_LED_Max) and the minimum current (I_LED_Min). For example, as shown in FIG. 4B, the final calibrated setting 409 can comprise a current I_LED setting located approximately midway (50%) between the maximum current (I_LED_Max) and the minimum current (I_LED_Min). The order of running the DRY and WET calibrations can be reversed.

In the embodiment of FIG. 4B, the method 500 may include, via operation of the controller (state machine) 310, setting the light emitter (e.g., light emitter 311) to operate at the selected final calibrated setting (e.g., final calibrated setting 409). In particular, the final calibrated setting 409 can correspond to the setting that can occur midway (50%) between I_LED_Max and I_LED_Min.

The method 500 can further comprise setting a comparator threshold (e.g., V_Ref_Final 409) based on selected ones of the first and second outputs. In particular, the method 500 can set the threshold voltage (e.g., V_{TH}) to a voltage value equal to V_Ref_Final. In other embodiments, V_{TH} can be set to a voltage value slightly above or below V_Ref_Final, such as +/−1 V_Ref division. V_{TH} for the FIGS. 3A and 4B embodiment can be set based on the midpoint between the intersection points E and F between the final calibrated setting line 409 and each of the WET curve 404B and DRY curve 402B.

The magnitude of the respective plurality of V_Ref inputs for the second embodiment can range from a minimum to a maximum value (e.g., from 0 to 15 V_Ref increments). The plurality of inputs for each embodiment can include at least 10 different input settings (e.g., % duty settings or V_Ref settings) which generate a variable light source current that is steady state once finalized. V_{TH} can then be selected for the final calibrated setting in each embodiment thus yielding maximum signal separation (FIGS. 4A and 4B).

For the second embodiment, a 2-step process can be employed (see FIG. 4B). Here the light emitter 311 can be pulsed at a 12.5% duty setting, but the current can be provided as a 10-bit variable signal from 3 mA to 85 mA (always at this fixed % duty cycle setting for the PWM). I_LED_FINAL 409 is provided based on a V_REF_FINAL 407 that is carefully selected from a choice of a plurality of discrete levels (e.g., 16 discreet levels from 1 to 15) to allow a WET state/DRY state discrimination with a comparator threshold V_{TH} that is approximately centered in the possible operating band between I_LED_Max and I_LED_Min. Both embodiments achieve the same end goal of achieving maximum noise immunity by separating the WET and DRY outputs as much as possible and centering the V_{TH} setting as much as possible.

Once calibrated for an individual supply line tubing 105, positioning, and liquid containing (WET) and non-liquid containing (DRY) modes, then can comprise operating the light emitter 311 at an intensity corresponding to the final calibrated setting (e.g., final calibrated setting 407) in order to accurately detect the supply line tubing 105 having no liquid therein, as well as the supply line tubing 105 having a liquid therein. If the output from the light detector 312 in output line 317 is equal to or above the threshold, then the supply line tubing 105 is determined by the controller (state machine) 310 to be WET (having the liquid therein), whereas if the output from the light detector 312 in output line 317 is below the threshold, then the supply line tubing 105 is determined to be DRY (having no liquid therein).

In each embodiment, the method can comprise storing, in a non-volatile memory 310M of the controller 210, 310, a value representing the threshold (e.g., a voltage V_{TH}). The threshold value can be used in conjunction with a generated operating parameter (e.g., voltage 409 or I_LED 409) that is: 1) at a maximum voltage difference (ΔV Max) between the first plurality of voltages 404A and the second plurality of voltages 402A, for the first embodiment, or 2) a (V_Ref) selected in conjunction with I_LED 409 located between the maximum current input (I_LED_Max) and the minimum current input (I_LED_Min), respectively, of the first plurality of currents 404B and the second plurality of currents 402B for the second embodiment.

Some of the above process blocks of method 500 may be executed or performed in an order or sequence not limited to the order and sequence shown and described. For example, in some embodiments, process block 504 may be performed before process block 502.

FIG. 6A illustrates a flowchart showing a generic method 600A of calibrating an optical bubble detector (e.g., bubble detector 202, 302) in more detail in accordance with one or more embodiments of the disclosure. The method 600A starts at process block 601 by turning the unit (e.g., bubble detector assembly 100, 300) ON. At decision block 603, the desired calibration mode is selected (WET calibration mode or DRY calibration mode). For example, the calibration mode can be selected by actuating (e.g., holding down) a calibrate button (e.g., calibrate button 308), which may be any suitable switch. Optionally, it may occur in an automatic sequence (e.g., DRY then WET calibration) once a calibration start (CALIBRATE) command is received, or vice versa. A calibration LED (e.g., calibration LED 204) may illuminate as the bubble detector assembly (e.g., bubble detector assembly 100) performs the calibration/self-tuning method 500. Other mechanisms or methods for starting DRY and WET calibrations may be used.

The start of the WET or DRY calibration modes may be optionally confirmed in either of process blocks 605 by receiving a signal on output line 217 or 317. The outputs acquired can be calibration data (Cal data) values in block 607D when conducting the DRY calibration mode or WET calibration data (Cal data) values in block 607W for each respective ones of the inputs (e.g., % duty settings or other input values) supplied on input line 215, 315. In the first embodiment, the inputs can be PWM signals having different % duty settings. For example, the ON duration of the various supplied % duty settings may increase in increments of 1% or 2% from 0% to a maximum % in the desired range. Other suitable increments may be used. The output data can be a plurality of voltages wherein each represents an amount of light detected through the supply line tubing 105 having no liquid therein (i.e., DRY) and then having liquid therein (i.e., WET). Each of the DRY and WET calibration values in output lines 217, 317 correspond respectively to inputs of the plurality of inputs to the light emitter 311 provided in input line 215, 315.

Each of the WET and DRY calibration mode values can be stored in memory 210M, 310M, such as NVRAM or other suitable memory type and/or may be forwarded to an external processor, microprocessor or computer (e.g., computer 316). Once the DRY and WET cal data points are acquired in blocks 607D, 607W, the method 600A can then optionally confirm to the user, in process blocks 609 that each of the DRY and the WET data collections have been individually completed. Process blocks 611 (if optionally used) can further verify that the collected calibration data points are monotonically increasing, for example, and that both DRY and WET calibrations have been completed. If increasing inputs of % duty settings (first embodiment) or increasing I_LED (second embodiment) does not yield increasing output in lines 217, 317 then something is malfunctioning and the whole routine can be aborted.

The method 600A, via controller 210, 310 (or other computer (e.g., computer 316) connected thereto), can then perform an analysis on the DRY and WET cal data points acquired and stored in a memory (e.g., in memory 210M or another memory) in process block 613. The analysis involves determining which one of the pluralities of inputs in line 215, 315 provides excellent signal separation between WET and DRY. For the first embodiment, the input 407 that causes a maximum voltage separation (ΔV Max between DRY data 404A and WET data 402A) is sought. In the second embodiment, a particular V_Ref value 407 is sought that causes an equal voltage difference between I_LED_Max for the WET data and I_LED_Min for the DRY data is sought.

After the analysis in block 613 is completed, the method 600A can perform an optional data check in block 615 to query whether is the data collected is adequate. This may test whether the maximum voltage separation (ΔV_Max) is above a preselected value, or that the input (I_LED_Min) is above a preselected minimum value. For example, a maximum voltage separation of at least 100 mV may be desired, or a minimum current (I_LED_Min) of 10 mA may be desired. If the data is not adequate (N), then there may be inadequate signal strength to keep the signal-to-noise ratio high or to establish a proper comparator reference. When the data is adequate (Y), in block 621, the particular input setting (the “final calibrated setting”) that causes the desired output is stored in memory 210M, 310M and is used for WET and DRY discrimination by the bubble detector assembly 100, 300 including the bubble detector 202, 302 going forward in a run mode (after completion of the self-calibration method 500).

Further, in block 621, a threshold may be selected as a comparator threshold that determines whether a WET or DRY condition exists going forward when in the run mode (non-calibration mode). The selection can be determined, for example, as V_{TH} in some embodiments. For example, in the first embodiment, the threshold V_{TH} can be a midpoint of the maximum voltage separation (ΔV_Max) and used as the comparator threshold V_{TH} going forward in the run mode. This comparator threshold V_{TH} can be saved to memory 210M. In some embodiments, the comparator threshold V_{TH} is the V_Ref_Final setting that is carefully chosen from the preselected settings. Here too, the value can be chosen as close to the midpoint between the intersections E and F of line 409 with the WET and DRY data of curves 404B, 402B as possible resulting in value 407. Different thresholds other than these midpoint values may be used.

As shown in block 623, the bubble detector assembly 100, 300 including the bubble detector 202, 302 is now calibrated and can be operable in the run mode. For example, the DRY/WET tubing indicator 306 can now be “ON” for a DRY condition in supply line tubing 105 and “OFF” for a WET condition in the supply line tubing 105 (or vice versa). Other mechanisms for flagging the WET or DRY condition may be used, such as sending a WET or DRY signal to a computer (e.g., computer 316) interconnected to the bubble detector assembly 100, 300.

FIG. 6B illustrates a flowchart showing a method 600B of calibrating a bubble detector assembly 100 including a bubble detector 202 as shown in FIG. 2 in accordance with the disclosure. The method 600B starts at process block 601 by turning the unit (e.g., bubble detector assembly 100) ON, as previously described. At decision block 603, the desired calibration mode (DRY calibration mode or WET calibration mode) can be selected. For example, the calibration mode can be selected by actuating (e.g., holding down) the calibrate button 208, which may be any suitable switch. Calibration LED 204 may illuminate as the bubble detector assembly 100 performs the calibration method 600B. For example, 1 blink of the LED 204 can indicate DRY calibration to be conducted, whereas 2 blinks can indicate the WET calibration to be conducted. The calibrate button 208 can be released right after desired mode (WET or DRY) is indicated. In one example, one periodic slow blink can indicate DRY calibration mode is being entered whereas two periodic slow blinks can indicate a WET calibration mode is being entered. Other suitable indications may be employed.

Assuming the first calibration mode entered is the DRY calibration mode, the press of button 208 can initiate issuance of the plurality of inputs as % duty settings from the controller 210 as well as acquisition of the resulting associated output voltages from the light detector 212. The output voltages can be provided as an A/D input to the controller 210 in block 607D. All of these digitized DRY calibration values can be stored in an array inside the controller 210 (e.g., microcontroller). The voltage values acquired can be V_DRY values in block 607D when conducting the DRY calibration for each respective ones of the % duty settings of PWM signals supplied in input line 215, each having different % duty settings. The V_DRY values can be referred to herein as the first plurality of voltages, wherein each represents an amount of light detected through the supply line tubing 105 having no liquid therein (i.e., DRY). Each of the V_DRY data values in output line 217 correspond respectively to inputs of the plurality of % duty settings to the light emitter 211 from input line 215.

When the second entered calibration mode is the WET calibration mode, the same plurality of inputs of % duty settings are issued from the controller 210. Now, however, the acquired V_WET output voltages in 607W lie on a different curve since the medium inside the supply line tubing 105 will have changed thus providing a comparatively different index of refraction. All of these digitized WET calibration values can be stored in an array inside the controller 210. The V_WET values in output line 217 can be a second plurality of voltages, for example.

Prior to acquiring the calibration data in each of the DRY and WET cases, the passage of air or liquid can be optionally confirmed in either of process blocks 605 by the operator or by other suitable means. Once the V_DRY and V_WET data (calibration data) is acquired, the method 600B can then optionally confirm separately to the user, in process blocks 609, via blinks (e.g., three blinks) of the calibration LED 204 or other suitable means, that the DRY and the WET data collections have been separately completed. The method 600B can then determine, in process blocks 611, that both of the DRY and WET calibrations (CALS) have been completed.

Once completed, the method 600B, via controller 210 or an external controller (e.g., external processor, microprocessor, or computer) interconnected to controller 210, can then perform an analysis on the V_DRY and V_WET data in process block 613. The V_DRY and V_WET data can be stored in memory 210M and/or forwarded and analyzed by controller 210 or sent to an external controller (e.g., external processor or computer) for analysis. In some embodiments, some portion of the analysis may be conducted by the controller 210, while other portions may be carried out by an external controller.

The analysis can comprise, in this first embodiment (the “max difference” embodiment) determining which one of the plurality of input of % duty settings provided in line 215 caused a maximum voltage separation (the maximum difference between V_WET and V_DRY). This may involve calculating a difference between the outputs of V_WET and V_DRY (ΔV=V_WET-V_DRY) for each input of a PWM signal with a different % duty setting. The analysis then compares each difference ΔV in order to select a maximum difference ΔV_Max between the respective WET and DRY data pairs. The output of light detector 212 can be connected to an analog input pin of the controller 210 where the signal can be digitized and read into a maximum difference-determining program configured to derive ΔV_Max. The maximum difference-seeking program may be a routine that is executed locally on the controller 210 or externally on a suitable controller (e.g., processor, microprocessor, or computer).

After the analysis in block 613 is completed, the method 600B can optionally query whether is there adequate voltage separation (ΔV_Max) to compute a maximum separation in block 615. For example, a voltage separation ΔV_Max of at least 100 mV may be desired. If sufficient voltage separation ΔV_Max is optionally present (Y), then three blinks (e.g., periodic slow blinks), or other suitable indication, may be sent to the calibration LED 204 to signify and confirm this. If the optional query of voltage separation ΔV_Max is not adequate (N), then continuous fast blinks, or another suitable indication, may be sent to the calibration LED 204 to signify this error condition. When the voltage separation (ΔV) is not adequate (N), there may be inadequate signal strength to keep the signal-to-noise ratio high or to establish a suitable comparator reference.

When the voltage separation ΔV_Max is adequate (Y), in block 621, the particular % duty setting (the “final calibrated setting”), i.e., the particular % duty setting that causes the maximum difference ΔV_Max can be stored in memory 210M and can then be used as an input for WET and DRY determinations by the bubble detector assembly 100 including the bubble detector 202 going forward in a run mode after the self-calibration method 600B.

Furthermore, in block 621, a threshold voltage may be selected. The selection can be calculated, for example, as a midpoint of the maximum voltage separation (ΔV_Max) and used as a comparator threshold V_{TH} reference going forward to determine the trigger point between WET and DRY determinations. This comparator threshold V_{TH} can be saved to memory 210M. Other comparator thresholds V_{TH} other than the midpoint may be used. For example, the comparator threshold V_{TH} may be 0.48× midpoint, or another fraction of ΔV_Max, such as +/−5% of the midpoint. This midpoint voltage as the comparator threshold may be used as the firmware program's comparator threshold V_{TH} for setting its output status LED to an “ON” state for DRY or an “OFF” state for WET (or vice versa).

As shown in block 623, the bubble detector assembly 100 including the bubble detector 202 is now calibrated and is operable in the run mode. As shown in FIG. 4A, if any measured voltage is equal to or above the threshold voltage V_{TH} (line 409) then it is determined to be WET condition in the supply line tubing 105, whereas below line 409 is determined to be DRY condition.

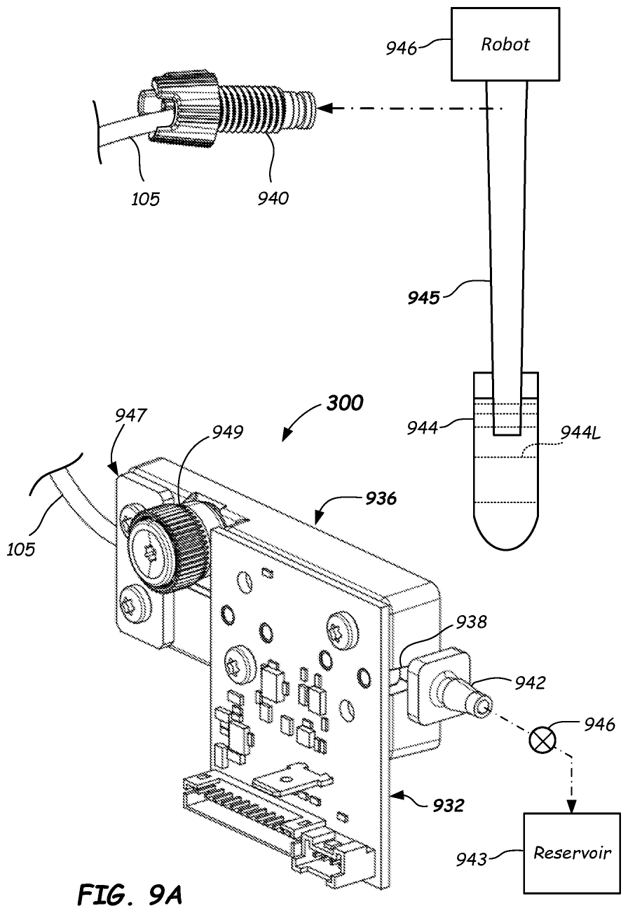

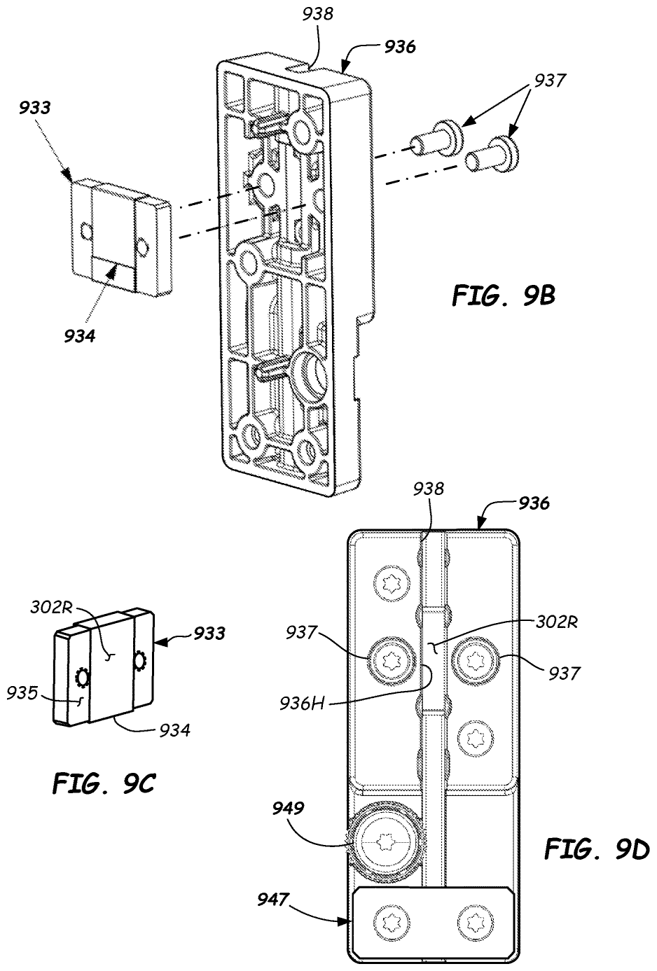

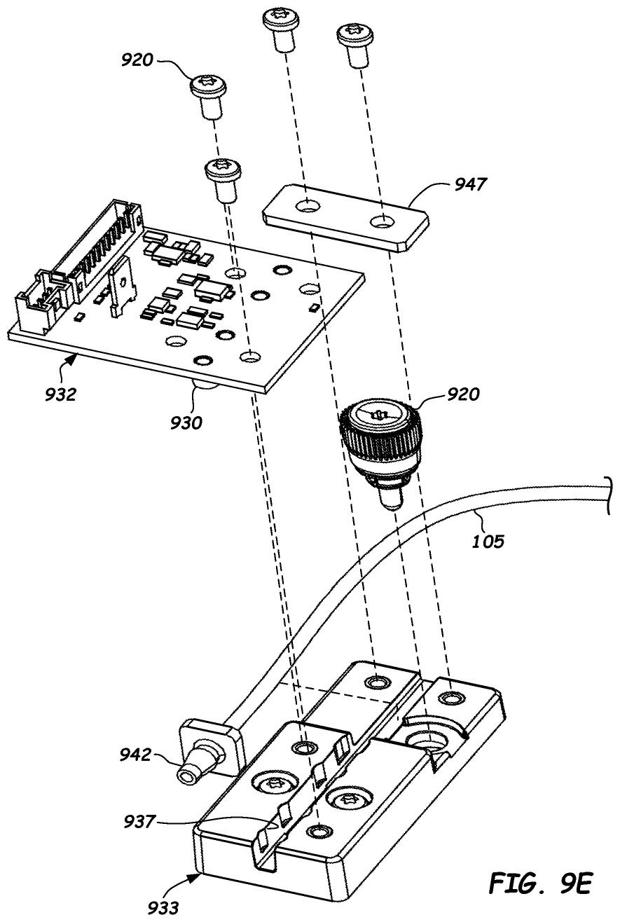

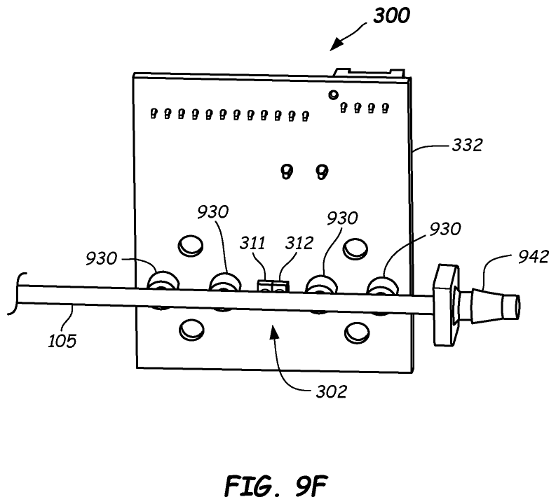

FIG. 6C illustrates a flowchart of a second method 600C of calibrating an optical bubble assembly 300 in more detail. This method 600C is referred to herein as the ½ Max-Min method herein. This second method 600C may be implemented by bubble detector assembly 300 of FIGS. 3A-3B and FIG. 9A-9F herein. In this method embodiment, a reflective-type bubble detector 302 is used to optically sense the difference between a DRY condition of the supply line tubing 105 (having no liquid therein) and a WET condition of the supply line tubing 105 (having a liquid therein).

This second method 600C starts at process block 601 by turning the unit (e.g., bubble detector assembly 300) ON as before. At decision block 603, the desired calibration mode is selected (DRY calibration mode or WET calibration mode). For example, the calibration mode can be selected by sending a calibration command (CALIBRATE) to the bubble detector assembly 300 or other suitable starting method, such as through base I/O board 303 from an interconnected external computer 316. Optionally, the selection order may occur in an automatic sequence (e.g., DRY then WET calibration) in some embodiments once the calibration start command is received, or vice versa. A confirmation signal in block 605 may be received, such as by base I/O board 303, once the bubble detector assembly (e.g., bubble detector assembly 300) successfully completes the calibration method 600C.

The start of the WET or DRY calibration modes includes passing either air or the liquid (e.g., liquid reagent, wash liquid, or process liquid) through the supply line tubing 105 at the location of the bubble detector 302. The optional confirmation in block 605 may be accomplished by receiving a test signal, such as by measuring supply line pressure, or other suitable means. For each of the DRY and WET calibration modes, I_LED data is acquired in blocks 607D and 607W. The outputs acquired can be calibration data (Cal data) values in block 607D when conducting the DRY calibration mode for each respective ones of the corresponding inputs supplied on input line 315. V_Ref values can range from a low voltage to a high voltage (e.g., from 245 mV to 676 mV), for example. The drive current to the light emitter 311 can range from about 3 mA to about 85 mA, for example. In this method 600C, the calibration is performed at each of the reference voltage (V_Ref) values that are available for use. In this manner a multi-point method is provided that sweeps across the available reference voltage inputs in order to generate the voltage output data that make up the WET and DRY calibration curves.

As shown, 15 different V_Ref levels are provided, being used as comparator voltage values that are incremented in magnitude from a lowest value to the highest value in approximately equal increments during each of the calibration modes (WET and DRY calibrations). Each V_Ref setting is used as a target voltage by the controller (state machine) 310 as the current is ramped from 3 mA to 85 mA during each calibration. The light receiver 312 receives the reflected light and provides a voltage feedback to the controller 310 via output line 317 and comparator 318. Once the climbing feedback output voltage at output line 317 reaches the target voltage V_Ref value for that increment, the associated I_LED is recorded.

The output data in output line 317 can be a plurality of voltages (or currents), wherein each output represents an amount of light detected through the supply line tubing 105 having no liquid therein (i.e., DRY calibration) or when having liquid therein (i.e., WET calibration) at each V_Ref setting. Each of the DRY calibration values in output line 317 can be a first plurality of outputs that correspond respectively to the plurality of inputs to the light emitter 311 from input line 315.

The wet calibration values in output line 317 can be a second plurality of outputs, wherein each represents an amount of light detected through the supply line tubing 105 having the liquid therein (i.e., WET calibration). Each of the Cal data values acquired as outputs in block 607W correspond respectively to the plurality of inputs to the light emitter 311 from input line 315. Each of the WET and DRY calibration mode values can be stored in memory 310M, such as NVRAM or other suitable memory type and/or sent to the computer 316.

Once the DRY and WET cal data points are acquired in blocks 607D, 607W, the method 600C can then optionally confirm individually, in process blocks 609, that each of the DRY and the WET data collections are completed. The method 600C can then determine, in process blocks 611, that both of the DRY and WET calibrations have been completed.

The method 600C, via controller 310 or computer 316, can then execute and perform an analysis on the DRY and WET cal data points acquired and stored in a memory (e.g., in memory 310M or in a memory of computer 316) in process block 613. The analysis can be carried out in computer 316 or controller 310 in order to determine which one of the plurality of inputs in line 315 provides excellent signal separation between WET and DRY conditions. For example, in this embodiment, once the final calibrated setting I_LED_FINAL is determined, the maximum V_Ref separation (as shown in FIG. 4B) between the DRY and WET data can be determined.

After the analysis in block 613 is completed, the method 600C can perform an optional data check in block 615 to query whether the data collected is adequate. This may test whether the I_LED_Min is above a preselected minimum current. For example, a minimum current (I_LED_Min) of 100 mA may be desired. If the data range is not adequate (N), then there may be inadequate signal strength to keep the signal-to-noise ratio high or to establish a suitable comparator reference.

When the data is adequate (Y), in block 621, the final calibrated setting (I_LED_Final) that causes the desired output is stored in memory 310M and is used as the current supplied to the light emitter 311 for WET and DRY determinations by the bubble detector assembly 300 including the bubble detector 302 going forward after the self-calibration. As discussed herein, I_LED_Final can be calculated to be equal to (I_LED_Max+I_LED_Min)/2. From I_LED_Final, the corresponding V_Ref can be estimated.

Further, in block 621, a comparator threshold may be selected that determines whether a WET or DRY condition exists going forward when in the run mode (non-calibration mode). The selection can be determined, for example, as V_{TH} in some embodiments. For example V_{TH} can be equal to V_REF_Final. V_REF_Final can be a midpoint 407 of the measured voltage separation (V_REF_Final=(WetVrefIntercept+DryVrefIntercept)/2) and used as a comparator threshold V_{TH} going forward in the run mode. This comparator threshold V_{TH} can be saved to memory 310M or other suitable memory. As shown on FIG. 4B, the comparator threshold V_{TH} may be set at a voltage located midway between WetVrefIntercept E and DryVrefIntercept F. Other comparator thresholds V_{TH} other than the midpoint value may be used, such as some other fraction of V_Ref_Final. The V_REF_Final value selected can be a V_Ref increment that is closest to the midpoint value.

As shown in block 623, the bubble detector assembly 300 including the bubble detector 302 is now calibrated and is now capable of operating in the run mode. The DRY/WET tubing indicator 306 can now be “ON” for a DRY condition detected in supply line tubing 105 and “OFF” for a WET condition detected in the supply line tubing 105 in the run mode (or vice versa). Other mechanisms for flagging the WET or DRY condition may be used, such as sending a WET or DRY signal to a base I/O board 303 or other controller (e.g., microprocessor, processor, or computer 316) interconnected to the bubble detector assembly 300.

FIGS. 9A-9E illustrate various views of an embodiment of a bubble detector assembly 300 including the optical bubble detector 302 and components thereof in accordance with one or more embodiments. The supply line tubing 105 (shown cut for illustration purposes in FIGS. 9A, 9E, and 9F) is disposed in a defined position relative to the bubble detector 302, as will be described herein. Bubble detector assembly 300 may include a calibration LED to display when calibration is underway and/or a WET/DRY indicator (e.g., WET/DRY indicator LED 306 as shown in FIG. 3B). Optionally, all the status indications, as well as data compilations may be provided through the SPP interface circuitry 301 to a base I/O board 303. Base I/O board 303 may be part of, or a peripheral of, a computer 316 that may be used to receive data and carry out analysis and may also provide input instructions and calibration commands to the bubble detector assembly 300 via the SPP interface circuitry 301.

The method 600C can be carried out by the bubble detector assembly 300 shown in FIGS. 3A and 3B and FIGS. 9A-9F. As was described above, the method 600C is operable to test (e.g., sweep) the bubble detector (e.g., bubble detector 302) across a range of reference voltage inputs to a light emitter 311 and to record the corresponding auto-calculated I_LED levels determined as being necessary to drive the light detector 312, to achieve the desired V_Ref values, which are shown in FIG. 4B. The auto-calculation of I_LED current for each V_Ref input setting is a process by which I_LED is ramped up from 3 mA to 85 mA until the photodiode output feeding the plus input of the comparator 319 is equal to the reference voltage (Vref) feeding the comparator's “-” input. Once the output of the light detector 312 reaches the level of V_Ref, the comparator output toggles from low to high. I_LED for that particular V_Ref is then latched into the NVRAM register or otherwise saved. This method is carried out at each of the V_Ref settings, with either the liquid or air inside the supply line tubing the whole time while all 16 V_Ref values (including zero) are being tested. These (V_Ref, I_LED) data pairs can be saved in the controller 310 and/or computer 316 and then may be analyzed in block 613 in order to determine a final calibrated setting and a comparator threshold V_{TH} in order to achieve excellent signal separation for the particular bubble detector 302 used; noting that each such bubble detector 302 can be slightly different from one another.

Once this analysis in block 613 is complete, values for I_LED_Final and the V_REF_Final may be written to memory (e.g., FLASH memory, such as an EEPROM 310M or other memory type) that may be part of the bubble detector assembly 300. Optionally, the data and analysis to determine such values for I_LED_Final and the V_REF_Final can be carried out by the controller (e.g., microprocessor, processor, or computer 316) interconnected to the base I/O board 303 (FIG. 3B) and external to the bubble detector assembly 300. Bubble detector assembly 300 may include an OPB9000 reflective optical sensor integrated circuit available from TT ELECTRONICS/OPTEK TECHNOLOGY, for example, as the adaptive sensor integrated circuit 307 (hereinafter “ASIC 307”).

The bubble detector assembly 300 (as well as bubble detector 302) can be configured to operate with an optically transparent or translucent tube, such as fluorinated ethylene propylene (FEP) material as the supply line tubing 105 or the like. The supply line tubing 105 can have dimensions such as an outer diameter of 2.54 mm, an inner diameter of 1.52 mm, and may have a wall thickness of 0.51 mm. However, the bubble detector 302 could be adapted to use other tubing sizes and types. The supply line tubing 105 may be fluidically connected at one end to a probe 945 by way of a connector 940 as shown in FIG. 9A. The other end of the supply line tubing 105 (shown truncated between connector 940 and the bubble detector assembly 300 for illustration purposes) may be connected to a liquid coupling 942 that is connectable to a distributor, valve, and/or pump, which in turn is coupled to a reservoir 943.

Depending upon use, the reservoir 943 can be a waste container (which may be held under vacuum) and a waste liquid can be aspirated by the probe 945 from a receptacle 944 (e.g., a cuvette) upon opening a valve or operating a pump. In the depicted embodiment, the connector 940 at the end of the supply line tubing 105 connects to the probe 945, which may be moveable by a robot 946 (e.g., including a stepper motor or the like) configured to control motion of the probe 945, such as by lowering the probe 945 into the receptacle 944 to evacuate the liquid 944L therein. Liquid may be wash liquid and/or process liquid.

In some embodiments, the connector 942 at the other end of the supply line tubing 105 can be connected to a flexible/collapsible tubing (e.g., a silicone-rubber tubing) that can be squeezed closed with a pinch valve 946. The tubing travels onward from the pinch valve 946 to the reservoir 943 (e.g., waste bottle) that may be held at a vacuum (e.g., negative pressure) in order to pull liquid 944L into and from the probe 945 when the pinch valve 946 is opened.

In other embodiments, the system may be configured to dispense a liquid (e.g., liquid reagent, wash liquid, or the like) into a receptacle 944, such as a cuvette. In such dispense operations, the bubble detector assembly 300 can be positioned directly proximate to the probe (like probe 945) so as to minimize a distance between the bubble detector 302 and the probe.

In more detail, the bubble detector assembly 300 is configured to provide a Wet/Dry output signal to determine whether the liquid is present (WET) or is not present (DRY) in the supply line tubing 105 at a location along the supply line tubing 105 where such a determination is desired. The output may be provided at J1 of the interface circuit 301 (see FIG. 3A) and may be carried by an interface cable 303C connected to the SPP interface 303I.

In some applications as discussed above, the bubble detector assembly 300 is designed to provide confirmation that a wash liquid has been aspirated and/or dispensed by the probe 945 located at a wash station, for example. The probe 945 and wash station can be part of a wash ring assembly of a diagnostic analyzer (e.g., immunoassay instrument or chemical analyzer). In other embodiments, a probe (like the probe 945 of FIG. 9A) can be part of a robot and probe assembly dispensing a liquid reagent into an incubation receptacle (e.g., cuvette or cup) residing in an incubation ring.

In more detail, the bubble detector assembly 300 can be fitted with a highly integrated optical reflective bubble detector 302 that is capable of detecting moving liquid and air slugs in the supply line tubing 105. The bubble detector 302 can have a very low (e.g., 6 μs) response time, for example. As will be apparent from the following, the bubble detector assembly 300 operates to auto-calibrate with the supply line tubing 105 used that is constrained in the specially designed mounting assembly as best shown in FIGS. 9A-9F.

Backing member 936 (FIGS. 9A and 9B) and standoffs 930 (FIGS. 9E-9F) of the assembly help to keep the supply line tubing 105 properly positioned and secured from moving relative to the bubble detector assembly 300 and further provides a highly reflective surface 302R affixed to a plate 933 behind the supply line tubing 105 to provide maximum light reflection to the light detector 312. The supplied light may be infrared (IR) light from the light emitter 311. The detected light can be amplified and compared to the determined threshold, thus providing a discrete comparator output. In this manner the bubble detector assembly 300 can discern between a WET or DRY condition inside the supply line tubing 105 in the run mode after calibration.

As shown in FIG. 3B, the functional operations of the bubble detector assembly 300 can be broken into the following functional blocks:
1) Serial peripheral port (SPP) interface circuitry 301 including ID EPROM 313,
2) Localized voltage regulation 305, and
3) ASIC 307.

Again referring to FIG. 3B, the simplified functional diagram of the bubble detector assembly 300 can include the SPP interface 301, which is designed to interface with a SPP port 303I of a base I/O board 303, which can interface with computer 316. The SPP interface 301 can operate to extend the functional features of the ASIC 307. J1 of FIG. 3A is an SPP interface connection to the ASIC 307 that can provide power and control thereof. The base I/O board 303 and the bubble detector board 932 (FIG. 9A) share an SPP interface 303I.

The base I/O board can manage high-level bubble detector functions over the SPP interface 303I. Low-level functions, such as auto-calculation of I_LED for a given V_Ref, can be managed by the ASIC 307. This ASIC 307 can have the discrete light emitter 311 (e.g., IR LED) and discrete IR light detector 312 (e.g., photodiode) mounted within it as described herein. A sensor sampling rate of 1 Hz to 1 KHz can be used. The bubble detector 302 includes an IR photo pair (e.g., light emitter 311 and light detector 312) aimed at the supply line tubing 105. The reflective surface or film 302R (FIGS. 9C-9D) can be positioned on an opposite side of the supply line tubing 105 from the light emitter 311 and light detector 312. As shown, reflective surface 302R can be suitably mounted to a backside of the backing member 936 and accessed through a hole 936H formed therein. The plotting of the WET and DRY calibration curves and the subsequent calculations performed on the data to determine I_LED_Final and V_REF_Final may be performed by the controller 310 and/or computer 316. The computer 316 can send commands such as SET_CONFIG, GET_CONFIG, READ_OUTPUT, and CALIBRATE to the ASIC 307 over the SPP interface circuit 301.

The output signal from the light detector 312 in output line 317 is a signal that is proportional to the reflected IR light, which changes intensity levels based on whether air (DRY) or liquid (WET) is contained in the supply line tubing 105. Power can be supplied from the base I/O board 303. The bubble detector assembly 300 can provide a 3.3V push-pull compatible logic or open-drain output. The discrete output logic can be configured to generate a HIGH for DRY and LOW for WET, or vice versa. The definitions for the pins of the SPP interface 301 are shown in Table 1 herein below.

TABLE 1
SPP Interface Pin Explanations

| | | Host | Host Mode | Host | | |
|---|---|---|---|---|---|---|
| Pin | SPP | Mode | Signal | Reset | Signal | SP |
| # | Signal | Used | Direction | State | Level | Implementation |
| 1 | 5.3 VDC | N/A | Power | Power | 5.3 VDC, ±5% | Power to SPP circuit. |
| | | | | ON | | 15 mA nominal |
| | | | | | | current used. |
| 2 | SER_A | GP | Output | Logic | 3.3 V | Used for RESET |
| | | OUTPUT | | High | CMOS | |
| 3 | SER_B | GPIO | Input | 10K | 3.3 V | Used as the |
| | | | | pull up | CMOS | DRY_WET_RAW |
| | | | | | | indicator |
| 4, 10 | GND | N/A | Power Return | N/A | 0 V | GND/RTN |
| 5 | 24 VDC | N/A | Power | Power | 24 VDC, ±10% | Not used |
| | | | | OFF | | |
| 6 | GPIO | GP | Output | High-Z | 3.3 V | Used for capturing |
| | | INPUT | | 20 uA | CMOS | autocalibration |
| | | | | pull | | confirmation pulse |
| | | | | down | | |
| 7 | SPI_DIN | SPI MISO | Input | 10K | 3.3 V | Used for capturing |
| | | | | pull up | CMOS | OPB9000 register |
| | | | | | | data and ID |
| | | | | | | EEPROM data |
| 8 | SPI_DOUT | SPI MOSI | Output | Logic | 3.3 V | Used for sending |
| | | | | High | CMOS | commands to |
| | | | | | | OPB9000 and ID |
| | | | | | | EEPROM |
| 9 | SPI_CLK | SPI | Output | Logic | 3.3 V | Used to clock ID |
| | | CLOCK | | High | CMOS | EEPROM |
| 11 | SPI_GENL | SPI | Output | Logic | 3.3 V | Used to steer signals |
| | | Device CS | | High | CMOS | in and out of |
| | | | | | | OPB9000 OUT and |
| | | | | | | CAL/STAT pins |
| 12 | SPI_ID_FLASH | SPI | Output | Logic | 3.3 V | Used to chip select |
| | | FLASH | | High | CMOS | ID EEPROM |
| | | CS | | | | |

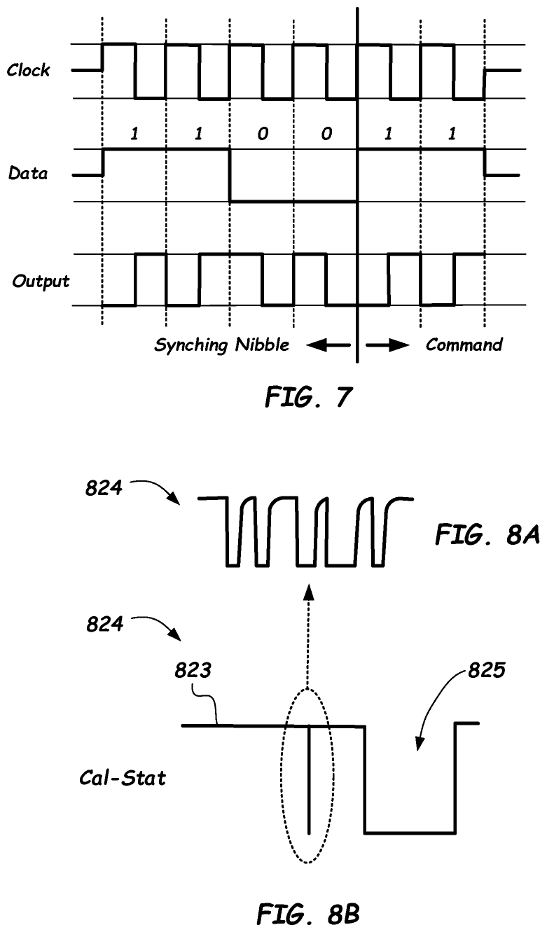

As is shown in Table 1 above, the supplied signal directions are with respect to the base SPP 303I. Communication with the bubble detector assembly 300 can be accomplished over the SPP interface circuitry 301 with a combination of SPI serial protocols and basic I/O polling methods. Although communication with the SPI EEPROM 310M can follow an SPI protocol, communication with the controller 310 (e.g., state machine shown) of the ASIC 307 can use a 1-wire interface and the Manchester protocol. FIG. 7 shows example encoding of a calibration request, and in particular the Manchester coding of the CALIBRATE command.

Communication with the registers of the ASIC 307 may be accomplished over this interface. EEPROM 310M can be a type of non-volatile memory that can be used to store calibration data and that can allow individual bytes to be erased and reprogrammed. It may also be used to store the PCA's identity and revision level. The state machine of controller 310 is hardware that can execute a behavior model and output several states. The model can include a finite number of states and is therefore also called finite-state machine (FSM). Based on the current state and a given input, the state machine performs state transitions and produces outputs.

The SPP interface circuit 301 brings power to the ASIC 307 and provides digital communications lines, as well. +5.3V power enters the SPP interface circuit 301, which can be immediately converted to +3.3V by a low dropout voltage regulator (LDO) 309. The LDO 309 is a type of power supply integrated circuit that can output a steady voltage from a supplied input voltage that may have some variability. The ASIC 307 can consume current of about 16 mA in operation.

The SPI communication and SPI GEN lines, labeled as part of the SPP interface circuitry 301 of FIG. 3B, are used to communicate with the ASIC 307 as well as with the ID EEPROM 313. SPI_CS_Flash is dedicated to the ID EEPROM 313. SPI_GENL is used to steer data to and from the ASIC 307. To understand this basic functioning, the ASIC 307 is explained below in detail.

ASIC 307 is a surface mount device (SMD) packaged integrated circuit (IC). Its function is to detect the presence or absence of an object via optical reflection. When powered and fitted with pull-up resistors attached to its OUT and Cal-Stat lines as shown in FIG. 3B, the ASIC 307 can provide a HI or LOW discrete output signal (from the OUT pin) depending on whether a reflective surface of a reflecting target is present or absent directly in front of it. In this diagnostic device application described herein, ASIC 307 can be configured for PUSH/PULL mode versus OPEN DRAIN MODE. All logic levels for I/O with the ASIC 307 are referenced to +3.3V.

Although ASIC 307 can run as-is, off the shelf, for basic operation with a robustly reflecting target article, usage of ASIC 307 with a less reflective surfaces, such as for detecting the presence of generally clear liquids in a supply line tubing 105, benefits from the special calibration provided by the inventive methods described herein. All communication with the ASIC 307 is through 2 digital pins, OUT and Cal-Stat. While the ASIC 307 functions as a sensor with discrete output from the OUT pin most of the time, it also allows access to internal registers via the Cal-Stat and OUT pins.

In most cases, serial commands are sent to the ASIC 307 via the Cal-Stat pin and received serially from the ASIC 307 via the OUT pin. When the ASIC 307 is not processing any commands, the OUT pin provides a function enabling output of a status of the bubble detection function, that is, HIGH for air and LOW for water. However when commands are being sent and the internal registers of the ASIC 307 are being accessed, the Cal-Stat pin receives WRITE, READ, and CALIBRATE commands. When a READ command is issued, the register contents can be dumped via the OUT pin. For the CALIBRATE command the ASIC 307 can generate a reply on the same pin, so this pin can be bi-directional.

Managing these complex communication scenarios can be handled by the use of multiple SPP digital control lines, a tristate buffer (TSB), and an analog multiplexer. Due to the serial nature of the incoming commands of the ASIC 307 and outgoing data, the SPI_DOUT controller line can be used to transmit to the Cal-Stat pin and the SPI_DIN controller line can receive data from the OUT pin, in tandem.

This steering can be accomplished in some embodiments using the tristate buffer IC (TSB) at the input to the Cal-Stat pin and an analog multiplexor IC at the output of the OUT pin. The SPI_GENL_chip select line can be used to manage the steering of these pathways. When SPI_GENL is LOW the SPI lines are connected to the ASIC 307 to support transmission of all serial commands to the ASIC 307 and receiving data from the ASIC 307 during the READ command. When SPI_GENL is HIGH then calibration confirmation data can pass from the Cal-Stat pin back to the controller 310 via the SPI_IO line and WET/DRY status data can pass from the OUT pin to the SER_B line. The latter condition can be set by the controller 310, for example. During normal operation, the only pathway in operation is the pathway created from the OUT pin to the SER_B line for communication of WET/DRY status. In this same signal steering state, the SPI-IO input is sampled by the controller 310 to detect the calibration confirmation pulse.

In the “auto-calibration mode” of operation, the SPI_GENL line can be strobed LOW via a signal from the base I/O board 303 and an auto-calibration command can be transmitted to the ASIC 307 over the Cal-Stat line through SPI_DOUT. The SPI_GENL LOW assertion also directs the OUT signal to the SPI_DIN pin, but it is ignored by the base I/O board 303 in this auto-calibration mode of operation. As soon as the calibration command transmission 824 (FIG. 8) is completed, the SPI-GENL line can be de-asserted HIGH in order to let the base I/O board 303 read back a confirmation pulse through SPI_DIN.

In a “manual programming mode” commands can be sent to set the current level of the light emitter 311, amplifier gain level, and output pin behavior. In this manual programming mode, SPI_GENL can be asserted LOW and both the Cal-Stat and OUT signals on the ASIC 307 can be simultaneously routed to the SPI_DOUT and SPI_DIN pins, respectively. The SPI-GENL stays LOW for both the transmission of the particular manual programming command and any ensuing response from the ASIC 307.

The SER_A pin is another SPP pin that can be used by the base I/O board 303 to conduct serial communications. Here, however, it can be used as a digital output. This digital output can allow power to be toggled ON or OFF for the entire board. WHEN SER_A is HIGH the board is powered up. When SER_A is LOW the board is powered down.

SPI_CS_FLASH can be used when the microcontroller on the base I/O board 303 is communicating with the ID EEPROM 313. ID EEPROM 313 can contain board identification information that includes the board type and version number. This can be queried by the microcontroller of the base I/O board 303 at initialization time and is used for decision making based on hardware features supported by the particular version number.

A microchip (e.g., a 1 Kbit, SPI Serial EEPROM) can be used as the ID EEPROM 313. This ID EEPROM 313 can be pre-programmed with a unique board type ID and board type ID revision. The board ID can be used by the particular diagnostic analyzer to confirm that the expected hardware configuration has been installed. The memory contents of the ID EEPROM 313 can be accessed via a simple SPI interface that uses SPI_CLK, SPI_DIN, SPI_DOUT and SPI_CS_FLSH signals for control.

Various measures can be used to minimize noise, cross-talk, and surge immunity problems. For example, +5.3V power can be passed through a power inductor before visiting the LDO 309 for improving power line immunity. Likewise, all I/O lines can have a low-pass filter in place (not shown). Finally, there can be transient voltage suppression applied to each I/O line (also not shown), to mitigate electrostatic discharge events.

In the case of the ASIC 307, the fully integrated, highly precise on-board circuitry, by using the methods herein, can distinguish between slightly different levels of reflected light. For this DRY/WET application, the ASIC 307 can be used to discern between the light reflected from a supply line tubing 105 that is either liquid filled (WET) or air-filled (DRY).

The ASIC 307 (e.g., OPB9000) can have features including an optical signal amplifier (which may be included in analog front end 318), output comparator 319, and finite state machine (State Machine operating as a controller 310) to control inputs to a DAC 320 and receive outputs from the light detector 312 so as to aid in running the auto-calibration method 600C described herein. OPB9000 can also incorporate other features such as light modulation for ambient light immunity and LED temperature compensation.

As discussed above, the ASIC 307 can use a Manchester serial communication protocol for communication, which can be a 1-wire protocol that uses a single line for clock and data. There are essentially only 3 commands, one of which allows an argument to be passed, such as READ, WRITE, and CALIBRATE. The WRITE can include a sequencing nibble and an argument that can be passed during the WRITE command. The READ command has no arguments since it dumps the contents of all registers. The CALIBRATE command also has no argument. An example of a coding command of a calibration request is shown in FIG. 8A.

The ASIC 307 can contain four nonvolatile registers: BANK1, BANK2, BANK3, and RESERVED. BANK3 and the RESERVED registers have 11 and 7 bits, respectively that are not documented. BANK2 has 6 bits, all of which are writable. The format for a WRITE to this register is 1101-10-bbbbbb. The bit level definitions here are: [SYNCING NIBBLE] [DETECTION NIBBLE]-[COMMAND NIBBLE]-[REF0] [REF1] [REF2] [REF3] [DS] [OP]. Four bits of this register are allocated for holding the reference level (REF0:REF3). The remaining two bits can be drain select (DS) and output polarity (OP), which can set the behavior of the discrete output pin. For drain select, 0 is set for open drain, 1 is set for push-pull operation. For output polarity, 0 is set for non-inverting, 1 is set for inverting.

BANK1 has 13 bits, of which 12 bits are writable. The format for a WRITE to this register is 1101-01-bbbbbbbbbbbbb. The bit level definitions here are: [SYNCING NIBBLE] [DETECTION NIBBLE]-[COMMAND NIBBLE]-[CA BIT] [AGC-LSB] [AGC-MSB] [LED0] [LED1] [LED2] [LED3] [LED4] [LED5] [LED6] [LED7] [LED8] [LED9] Bank 1 has 10 bits allocated to the LED current level, 2 bits allocated for automatic gain control (AGC) of the detected light, and 1 bit assigned for confirmation that calibration was successful (1=successful). AGC=00 sets a gain of 1, AGC=01 sets a gain of 4, AGC=10 sets a gain of 8. The programming communication rate for the WRITE command can be 100 kbit/s max at 50% duty setting. The programming communication rate for the READ command can be 100 kbit/s+5 kbit/s at 50% duty setting.

FIG. 8B illustrates a zoomed-out view (lower trace) of an auto-calibration command 823 that can be issued on the Cal-Stat line to initiate the auto-calibration method 600C described herein. FIG. 8A shows a zoomed-in view (upper trace) of a calibration request 824 that is sent from the microcontroller to the Cal Stat pin of the ASIC 307 during the CALIBRATE command 823. Upon receipt of this auto-calibration command 823 including the calibration request 824, the pulsed LED drive can ramp up its current at the preset V_Ref setting over a range of 3 mA to 85 mA. The resulting I_LED for each V_Ref can be recorded of the memory 310M (e.g., EEPROM) of the ASIC 307 as shown in FIG. 4B. The auto-calibration method 600C can take less than a second. If this method passes, then a 13 ms LOW pulse 825 can be transmitted out of the Cal-Stat PIN. Also the CA bit in BANK1 can be set high.

In more detail, the multi-point self-calibration method 600C is best shown in FIGS. 6C-6F. The method 600C herein operates to achieve calibration with improved WET/DRY signal separation. This, as stated before, is especially important because the signal differences between WET and DRY readings when trying to detect a clear or clearish liquid such as in diagnostic analyzers is very slight. In order to improve the WET versus DRY signal separation and achieve excellent noise immunity, this self-calibration method 600C generates the multi-point WET calibration curve 404B (e.g., a 16 point WET calibration curve) and a multi-point DRY calibration curve 402B (e.g., a 16 point DRY calibration curve), such as shown in FIG. 4B.

The method 600C involves, as shown in FIG. 6C, turning ON the electrical circuit 300B at block 601. This can be accomplished by powering the bubble detector assembly 300 via power from the base I/O board 303. Next, a calibration mode is selected in block 603. This can be done automatically, such as running DRY and then WET calibration, or vise-versa. Further, a CALIBRATE signal may be provided from the base I/O board 303, such as through the SPI_DOUT pin (FIG. 3B), to initiate calibration.

The method 600C then passes either the liquid (e.g., liquid reagent, wash liquid, or the like) or air through the supply line tubing 105, so that the liquid or air is present at the location of the bubble detector 302. The passing of the liquid or air can optionally be confirmed in blocks 605. The confirmation can take place by monitoring the pump, for example, or otherwise monitoring the pressure in the supply line tubing 105.

For each mode (WET calibration mode and DRY calibration mode) calibration data is acquired. In blocks 607D and 607W, I_LED data is obtained, as shown in FIG. 4B based on the incremental voltage inputs. The data acquired can be stored locally and/or may be passed on to the base I/O board 303 and ultimately passed further up to the computer 316 where the data can be plotted and/or analyzed. A graphical display of the data acquired (V_Ref vs. I_LED) is shown in FIG. 4B. Data for the DRY and WET curves 402B, 404B can be generated by ramping up I_LED via voltage inputs to DAC 320 in line 315 at each V_Ref setting until the detected and amplified light signal from light detector 311 reaches each V_Ref level (FIG. 4B). During the calibration, a pulsed LED drive signal from the DAC 320 is ramped up to result in a current to the light emitter 311 of from about 3 mA to about 85 mA. The total ramping period can be about 17 mS. The V_Ref values at which the calibration procedure is performed can vary in equally-spaced increments from 245 mV to 676 mV, represented in settings from 0 to 15, for example. However, other numbers of increments and ramping periods could be used.

During the calibration mode, the Cal-Stat pin can be initially asserted HIGH from the SPI_DOUT pin. Calibration is started via the CALIBRATE command routed to the Cal-Stat pin from the SPP interface 301, which is facilitated by setting SPI_GENL=0. In this mode, the tristate buffer TSB can drive command data from SPI_DOUT onto the Cal-Stat pin of the ASIC 307. As soon as the command transmission is complete the tristate buffer TSB is set to HI-Z mode. This allows for the ASIC 307 to drive the Cal-Stat pin. After a successful calibration, which can last about 17 ms, the Cal-Stat pin will transition to a LOW state for 13 ms. An unsuccessful calibration can occur if a reflective surface 302R is not present or there is insufficient reflected light received by the light detector 312 during the calibration. The DAC 320 used can be a 12-bit current control circuit that can sink the cathode of the LED to ground. The thick arrow entering the DAC 320, labeled 315, from the control logic (state machine) of the controller 310 can represent 10 current control+2 gain control digital lines that pass from the controller 310 to the DAC 320 to control the ramping up of the current to the light emitter 311. The pulse digital line (thinner line) that passes from the controller 310A to the DAC 320 is a gate drive that modulates the DAC 320 on and off. When this digitally-controlled gate drive is in its ON-duty phase prescribed current sink level selected by the DAC 320 is issued. In its OFF-duty phase, there is no current sink occurring.

The current to the light emitter 311 (LED) can be pulsed at 500 KHz, 12.5% duty. In this embodiment, variability of the intensity of the light emitter 311 (e.g., LED) is not being controlled by PWM signal as in the FIG. 2 embodiment, rather, it is controlled independently via opening or closing the channel of the drive to admit or restrict current flow to the light emitter 311.

Upon passing through the supply line tubing 105 a first time, being reflected off from the reflective surface 320R, and passing through the supply line tubing 105 a second time, the remaining un-scattered light from the light emitter 311 is received by the light detector 312, which may be any suitable photodiode. For any given V_Ref applied, increasing that voltage can provide an increase in current to the light emitter 311 and therefore induce an increase in the voltage from the light detector 312.

FIG. 6D illustrates a flowchart of a method of generating a DRY or WET calibration curve for calibration of the second bubble detector assembly 300. In this method the calibration is performed at each of the reference voltage values (V_Ref) that are available for use. In this manner a multi-point method is provided that sweeps across the available reference voltage inputs and feeds into an internal comparator 319 of the optical bubble detector assembly 300 according to embodiments. In particular, FIG. 6D illustrates one method of how the I_LED values can be acquired in blocks 607D and 607W.

In block 608, the value of V_Ref is set to zero at the beginning of the sweep. In block 610, the calibration is performed by sweeping V_Ref from a minimum value of V_Ref Min=0 to a maximum value of V_Ref_Max. The values of V_Ref can be integer values. V_Ref_Max can be an integer number, such as 15. So in this example, the V_Ref is swept and incremented by DAC 320 from 0 to 15 in increments of one, wherein each number is proportional to the voltage output in FIG. 3B. As V_Ref is being swept, an I_LED value generated from the current received from light detector 312 in output line 317 necessary to match each V_Ref value, as determined by comparator 319, is recorded in block 612. For example, the resulting I_LED values corresponding to each V_Ref increment may be stored in local memory. Optionally or additionally, the data may be sent to the base I/O board 303 and to computer 316.

The outputs in line 317 from the light detector 312 that correspond to each incremental V_Ref provided as an input to comparator 319 can be received by an analog front end 318. The analog front end 318 may be bandpass-filtered in order to phase lock the photodetector circuitry to the LED drive circuitry. So the output signal in 317 is only received during a time interval when the gate drive signal of the DAC 320 is enabled. In this manner, any optical signal that is constant (like ambient light) or other noise artifacts that are occurring at other frequencies (e.g., at 60 Hz noise) can be ignored. Analog front end 318 may also include suitable amplification.

When the V_Ref is equal to V_Ref_Max in block 614, then the incrementing stops for the DRY calibration mode and the WET calibration mode can start by repeating the steps in FIG. 6D for the WET calibration mode. In other embodiments WET calibration can occur before DRY calibration.

Again referring to FIG. 6C, once the data sweep in FIG. 6D is completed, the self-calibration method 600C can optionally implement confirmations in blocks 609 to ensure that sufficient data has been generated individually for the WET and DRY calibrations. This confirmation can be achieved by the controller 310 assessing that there were the desired number (e.g., 15) of successful calibrations in a row, one for each V_Ref input. Next, in block 613, the self-calibration method 600C can perform an analysis of the data points making up the WET and DRY calibration curves 404B, 402B that were obtained and stored from implementing the method of FIG. 6D in order to obtain final values for I_LED_Final and V_Ref_Final. As a precursor to, or at the same time as the analysis, an optional data check can be performed in block 615 to see if the data received will be adequate for analysis. For example, the data may be checked to see that the maximum I_LED received has at least met a minimum pre-established current level, such as 10 mA, or to evaluate the value of the difference between the V_Ref values at the intersection points with I_LED line 409 to a pre-established voltage delta value (ΔV). In particular, the pre-established voltage delta value (ΔV) should be 3.50 or more full V_REF divisions between the WET calibration curve 404B and DRY calibration curve 402B at I_LED_Final 409. Additionally or optionally, the I_DRY/I_WET current ratio (C1/C2) at V_Ref_Final on line 407 should be C1/C1≥1.15.

As shown in block 621, the analysis of the method 600C in block 613 finally results in a determination of an I_LED_Final, which is a current value that can be stored in the EEPROM 310M. In short, the analysis in block 613 finds as a final calibrated setting 409, i.e., a current setting, that results in an I_LED current to the light emitter 311 that allows suitable operation within the active region of both WET (404B) and DRY (402B) calibration data curves.

To aid in understanding how the final calibrated setting 409 may be chosen in this embodiment, reference is made to FIG. 4B and FIG. 6E. The calibrated I_LED_Final setting method 613I shown in FIG. 6E comprises, in block 620, finding I_LED_Max as the maximum current value on the WET curve 404B. In block 622, the method 613I comprises finding I_LED_Min as the minimum current value on the DRY curve 402B. In block 624, the I_LED_Final value, i.e., the final calibrated setting, is determined. This I_LED_Final value is used for WET/DRY detection going forward as shown in Block 621 (FIG. 6C). The I_LED_Final value can be calculated as: (I_LED_Max+I_LED_Min)/2, for example. This I_LED_Final value can be rounded to the nearest whole number corresponding to a V_Ref value that the DAC 320 is capable of outputting.

According to the method 600C, the analysis in block 613 on the V_DRY and V_WET data can further determine a final reference value V_Ref_Final, which is a comparator threshold going forward when determining the WET or DRY determination of the supply line tubing 105. One embodiment, a threshold determining method 613T for determining V_Ref_Final is shown in FIG. 6F.

Knowing the I_LED_Final from the previous flowchart of FIG. 6E, V_Ref_Final can now be determined. I_LED_Final can be rounded to the nearest value. According to the method 613T, in block 626, the WetV_RefIntercept (point E) on FIG. 4B is calculated, and, in block 628, the DryV_RefIntercept (point F) is calculated. This involves identifying the wet straddle points (A and B) and the dry straddle points C and D. Wet straddle points, referred to herein as WetHighEdge (point A) and WetLowEdge (point B), straddle either side of the WetV_RefIntercept (point E) as shown in FIG. 4B. Similarly, Dry straddle points, referred to herein as DryHighEdge (point C) and DryLowEdge (point D), straddle either side of the DryV_RefIntercept (point F) as shown in FIG. 4B. The straddle points can be the points immediately above and below the intersection points E and F.

In block 630, V_Ref_Final is determined. V_Ref_Final can be determined by determining, in any order, WetV_Ref Intercept and DryV_RefIntercept, as follows:

As shown in block 626, WetV_RefIntercept (point E) is calculated using the following calculations and definitions:

WetHighEdge=The WET calibration current value that is closest to AND greater than or equal to the Final I_LED_Final at point A.

WetHighV_Ref=The V_Ref value that corresponds to the WetHighEdge current at point A.

WetLowEdge=The WET calibration current value that is closest to AND Less than or equal to the I_LED_Final at point B.

WetLowV_Ref=The V_Ref value that corresponds to the WetLowEdge current at point B.

WetYIntercept=The Y intercept of a straight line that passes through the points (WetHighV_Ref, WetHighEdge) and (WetLowV_Ref, WetLow Edge), i.e., through points A and B.

WetSlope=The slope of the line passing through the points (WetHighV_Ref, WetHighEdge) and (WetLowV_Ref, WetLowEdge), i.e., slope of line passing through points A and B.

WetV_RefIntercept=The point at which I_LED_Final current value intersects with the WET calibration curve 404B.

WetSlope=(WetHighEdge−WetLowEdge)/(WetHighV_Ref−WetLowV_Ref)=WetHighEdge−WetLowEdge/1=WetHighEdge−WetLowEdge (Note that the ΔV_Ref always equals 1)

WetYIntercept=WetLowEdge−(WetLowV_Ref+(WetHighEdge−WetLowEdge))

WetV_RefIntercept(point E)=(I_LED_Final−WetYIntercept)/(WetHighEdge−WetLowEdge)

If the I_LED_Final current intersects the wet calibration curve 404B directly on a calibration point (e.g., one of points 1-15) then WetV_RefIntercept is equal to the V_Ref value of that calibration point.

As shown in block 628, DryV_RefIntercept (point F) is calculated using the following calculations and definitions:

DryHighEdge=The Dry calibration current value that is closest to AND greater than or equal to the I_LED_Final current at point C.

DryHighV_Ref=The V_Ref value that corresponds to the DryHighEdge current at point C.

DryLowEdge=The dry calibration current value that is closest to AND Less than or equal to the I_LED_Final current at point D.

DryLowvRef=The V_Ref value that corresponds to the DryLowEdge current at point D.

DryYIntercept=The Y intercept of a straight line that would pass through the points (WetHighV_Ref, WetHighEdge) and (WetLowV_Ref, WetLowEdge), i.e., through points C and D.

DrySlope=The slope of the line passing through the points (DryHighV_Ref, DryHighEdge) and (DryLowV_Ref, DryLowEdge), i.e., slope of the line through points C and D.

DryV_RefIntercept=The V_Ref point at which I_LED_Final current value intersects with the WET calibration curve 404B.

Wet Slope=(DryHighEdge−DryLowEdge)/(DryHighV_Ref−DryLowV_Ref)=(DryHighEdge−DryLowEdge)/1=(DryHighEdge−DryLowEdge).(Note that the ΔV ref always equals1)

DryYIntercept=DryLowV_Ref*(DryHighEdge−DryLowEdge)

DryV_RefIntercept(F)=(I_LED_Final−DryYIntercept)/(DryHighEdge−DryLowEdge).

If the I_LED_Final current 409 intersects the dry calibration curve 402B on a calibration point then DryV_RefIntercept is equal to the V_Ref value of that calibration point.

The method 613T can optionally perform a DeltaV_Ref test for pass/fail in block 630. The optional test can analyze the V_Ref values of points E and F. The test can involve calculating a DeltaV_Ref value, wherein:

DeltaV_Ref=WetV_RefIntercept−DryV_RefIntercept.

The Pass/Fail Criteria can be a DeltaV_Ref value that is 3.50 or more full V_Ref divisions (point E-point F) between the intersection of WET calibration curve 404B and DRY calibration curve 402B at I_LED_Final 409.

In some embodiments, a current ratio (CR) test may also be optionally performed in block 632, wherein:
C1=The DRY calibration current value on DRY curve 402B that corresponds to V_Ref_Final, and
C2=The wet calibration current value on WET curve 404B that corresponds to V_Ref_Final.

The current ratio is defined as: CR=C1/C2. The Pass/Fail Criteria can be that CR at V_REF_Final must be ≥ 1.5, for example.

After determining the WetV_RefIntercept and the DryV_RefIntercept in blocks 626 and 628, V_Ref_Final value can be calculated in block 634, as follows:

V_Ref_Final=(WetV_RefIntercept+DryV_RefIntercept)/2

The V_Ref_Final value can be rounded to the nearest V_Ref integer. The V_Ref_Final value can be used as the comparator threshold V_{TH} going forward. The comparator threshold V_{TH} can be used as a demarcation (trigger) point between WET and DRY determinations. As should be recognized, the resulting value of V_Ref_Final is a reference voltage setting that achieves a highest level of signal separation between the WET and DRY states.

These two final parameters (I_LED_Final and V_Ref_Final) can be written and stored to the EEPROM 310M. This calibration method 600C of FIG. 6C allows the finding of an excellent trip point for signaling the DRY and WET states in use going forward.

Again referring to FIGS. 9A-9F, the ASIC 307 of the bubble detector assembly 300 can comprise standoffs 930, such as cylinders (e.g., metal cylinders) that can be coupled to a printed circuit board 932 that operate to contact and position the supply line tubing 105 at a defined distance away from the light emitter 311 and light detector 312 (FIG. 9E). For example, the defined distance between the light emitter 311 (or light detector 312) and the closest part of the supply line tubing 105 can be approximately 2.09 mm. However, other suitable distances can be used. On the other side of the supply line tubing 105, the reflective surface 302R is provided. The reflective surface 302R can be formed from any suitable reflective material, such as a reflective film 934. Reflective film 934 may be a 3M™ SCOTCHLITE™ reflective graphic film 680-10, or the like. Reflective film 934 may be affixed to a surface, such as a flat surface 935 of support member 933. For example, the reflective film 934 may be wrapped about the support member 933 and fastened thereto. Support member 933 may be a rigid block, such as an aluminum block, for example.

The support member 933 with reflective surface 302R may be coupled to the backing member 936, such as by using fasteners 937 as shown in FIGS. 9B and 9D or another suitable attachment mechanism. Backing member 936 can comprise a groove 938 along its length that is configured to receive the supply line tubing 105 therein. When the backing member 933 is assembled to the printed circuit board 932 as shown in FIG. 9A, the bottom of the groove 938 makes contact with the supply line tubing 105 along its length as do the standoffs 930 and thus the position of the supply line tubing 105 relative to both the light emitter 311 and light detector 312 and further to the reflective surface 302R can be precisely controlled. The reflective surface 302R may be configured to be approximately 2.5 mm away from the closest part of the supply line tubing 105. However, other suitable distances may be used.

The supply line tubing 105 may be further secured by bracket 947 coupled to backing member 936, as shown. An attachment fastener 949 may be part of bubble detector assembly 300 and used to secure the bubble detector assembly 300 to a structure. For example, in some embodiments, the bubble detector assembly 300 can be mounted to a frame of the diagnostic analyzer, to the probe 945 itself, to a structure (not shown) attached to the probe 945, to a part of a robot (not shown) operating to move the probe 945, or to a wash station.

In another embodiment, a computer program product is provided. The computer program product comprises a non-transitory medium readable by a computer, the computer readable medium having computer program code configured to: receive a first plurality of outputs each representing an amount of light detected through a supply line tubing having no liquid therein for a respective plurality of inputs to a light emitter; receive a second plurality of outputs each representing an amount of light detected through the supply line tubing having a liquid therein for the respective plurality of inputs; and set a threshold based on selected ones of the first plurality of outputs and the second plurality of outputs.

In one embodiment, the computer program code of the computer program product can be configured to select one of the respective plurality of inputs at which a largest difference occurs between a respective pair of the first and second pluralities of outputs, and determine a threshold based on the respective pair of the first and second pluralities of outputs at which the largest difference occurs. In another embodiment, the computer program code of the computer program product can be configured to select an operating input (I_LED_Final) based on a minimum I_LED value (I_LED_Min) and a maximum I_LED value (I_LED_Max). The threshold may be located midway between the intersection of the I_LED_Final with the WET and DRY I_LED vs V_Ref curves.

Persons skilled in the art should readily appreciate that the disclosure described herein is susceptible of broad utility and application is diagnostic analyzers. Many embodiments and adaptations of the disclosed embodiments other than those described herein, as well as many variations, modifications, and equivalent arrangements, will be apparent from, or reasonably suggested by, the disclosure and the detailed description herein, without departing from the substance or scope of the disclosure.

For example, although described in connection with a particular liquid bubble detector, one or more embodiments of the disclosure may be used with other types of liquid bubble detectors. Accordingly, while the disclosure has been described herein in detail in relation to specific embodiments, it should be understood that this disclosure is only illustrative, presents examples of the disclosure, and is made merely for purposes of providing a full and enabling disclosure. This disclosure is not intended to limit the invention to the particular apparatus, devices, assemblies, systems, or methods disclosed herein, but, to the contrary, the intention is to cover all modifications, equivalents, and alternatives falling within the scope of the claims.

What is claimed is:

1. Optical bubble detector assembly, comprising:
a controller configured to:
operate a light emitter projecting light into a supply line tubing of an optical bubble detector at a plurality of light intensities corresponding to a plurality of inputs;
receive a first plurality of outputs from a light detector of the optical bubble detector, the first plurality of outputs corresponding respectively to the plurality of inputs, each of the first plurality of outputs representing an amount of light detected through the supply line tubing having no liquid therein;
receive a second plurality of outputs from the light detector, the second plurality of outputs corresponding respectively to the plurality of inputs, each of the second plurality of outputs representing an amount of light detected through the supply line tubing having a liquid therein; and
select a particular one of the plurality of inputs as a final calibrated setting based upon selected ones of the first and second pluralities of outputs.

2. The assembly of claim 1, wherein the controller is further configured to determine a threshold based upon selected ones of the first and second pluralities of outputs.

3. The assembly of claim 1, wherein the controller is configured to operate the light emitter at the final calibrated setting when detecting whether the supply line tubing contains the liquid or air.

4. The assembly of claim 1, wherein the controller is further configured to:
select a maximum current based on the first plurality of outputs;
select a minimum current based on the second plurality of outputs; and
determine the final calibrated setting located between the maximum current and the minimum current.

5. The assembly of claim 4, wherein the final calibrated setting comprises a value located approximately midway between the maximum current and the minimum current.

6. The assembly of claim 1, wherein the controller is configured to:
select a threshold as a voltage value at which a largest voltage difference occurs between a respective pair of the first and second pluralities of voltages.

7. The assembly of claim 6, wherein the threshold comprises an operating voltage located midway between the respective pair of the first and second pluralities of voltages at which the largest voltage difference occurs.

8. The assembly of claim 1, wherein the controller is configured to calculate for each of a plurality of duty cycles a voltage difference between each respective pair of the first and second pluralities of outputs.

9. The assembly of claim 1, further comprising an operational amplifier coupled to receive a pulse width modulated input from the controller and configured to provide a current input to the light emitter in response thereto.

10. The assembly of claim 1, wherein the controller is configured to store in non-volatile memory a plurality of stored values representing the first and second pluralities of outputs.

11. The assembly of claim 1, wherein the controller is configured to operate the light emitting device at the final calibrated setting and determine whether a supply line tubing has a liquid or air therein based on a threshold.

12. The assembly of claim 1, wherein the supply line tubing is held between a backing member and standoffs to space the light emitter and light detector from the supply line tubing.

13. The assembly of claim 12, wherein the supply line tubing is held in a groove formed in the backing member.

14. A method of calibrating an optical bubble detector, comprising:
receiving a first plurality of outputs each representing an amount of light detected through a supply line tubing having no liquid therein, the first plurality of outputs corresponding respectively to a plurality of inputs to a light emitter;
receiving a second plurality of outputs each representing an amount of light detected through the supply line tubing having a liquid therein, the second plurality of outputs corresponding respectively to the plurality of inputs to the light emitter; and
selecting a particular one of the plurality of inputs as a final calibrated setting based upon selected ones of the first and second pluralities of outputs.

15. The method of claim 14, further comprising setting a threshold based on selected ones of the first and second pluralities of outputs.

16. The method of claim 15, wherein a controller is operational to:
select a minimum current based on the first plurality of outputs;
select a maximum current based on the second plurality of outputs; and
determine the final calibrated setting between the maximum current and the minimum current.

17. The method of claim 16, wherein the final calibrated setting is located midway between the maximum current and the minimum current

18. The method of claim 14, further comprising setting the light emitter to operate at a final calibrated setting corresponding to where a largest voltage difference occurs between a respective pair of the first plurality of outputs and the second plurality of outputs.

19. The method of claim 14, further comprising setting a threshold based on the respective pair of the first plurality of outputs and the second plurality of outputs.

20. The method of claim 14, further comprising calculating, with a controller, for each respective plurality of inputs a difference between each respective pair of the first plurality of outputs and the second plurality of outputs.

21. The method of claim 14, wherein the plurality of inputs are % duty settings over a range from 10% to 40% of a maximum output.

22. The method of claim 14, wherein the plurality of inputs comprise at least 10 different inputs.

23. The method of claim 14, further comprising operating the light emitter at the final calibrated setting to detect the supply line tubing having either no liquid therein or having the liquid therein.

24. The method of claim 14, further comprising storing, in a non-volatile memory, a value representing a threshold corresponding to an operating output located between the first plurality of outputs and the second plurality of outputs.

25. A computer program product, comprising:
a non-transitory medium readable by a computer, the computer readable medium having computer program code configured to:
receive a first plurality of outputs each representing an amount of light detected through a supply line tubing having no liquid therein for a respective plurality of inputs to a light emitter;
receive a second plurality of outputs each representing an amount of light detected through the supply line tubing having a liquid therein for the respective plurality of inputs; and
set a final calibrated setting based on selected ones of the first plurality of outputs and the second plurality of outputs.

26. The computer program product of claim 25, wherein the computer program code is configured to:
select one of the respective plurality of inputs at which a largest difference occurs between a respective pair of the first and second pluralities of outputs; and
determine the final calibrated setting based on the respective pair of the first and second pluralities of outputs at which the largest difference occurs.

27. The computer program product of claim 25, wherein the computer program code is configured to:
determine a minimum I_LED based on the first plurality of outputs;
determine a maximum I_LED based on the second plurality of outputs; and
select the final calibrated setting between the maximum I_LED and the minimum I_LED.

28. The computer program product of claim 27, wherein a threshold is determined based on intersection of the final calibrated setting with a WET curve and a DRY curve.
